(19) United States
(12) Patent Application Publication
(10) Pub. No.: US 2018/0179497 A1
(43) Pub. Date: Jun. 28, 2018

(54) Artificial immune cell emulating selected properties of granulocytes utilizing Fenton-type reactions

(71) Applicants: Nikita Balashov Katz, Salt Lake City, UT (US); Serghei Railean, Salt Lake City, UT (US)

(72) Inventors: Nikita Balashov Katz, Salt Lake City, UT (US); Serghei Railean, Salt Lake City, UT (US)

(21) Appl. No.: 15/849,606

(22) Filed: Dec. 20, 2017

Related U.S. Application Data
(60) Provisional application No. 62/436,450, filed on Dec. 20, 2016.

(51) Int. Cl. C12N 5/0787

(52) U.S. Cl. CPC C12N 5/0642; C11B 5/0085

(57) ABSTRACT

An artificial immune cell emulating certain properties of the granulocytes is disclosed as a construct or aggregate of several constituent particles that possess the properties of catalysis therefore producing free radicals and reactive species of oxygen, nitrogen, halogens and the like in the classical Fenton and Fenton-like reactions with produced free radicals serving as signaling molecules and, in higher concentrations, as toxic factors for microorganisms, cancerous cells, abnormal tissue and other biological targets, emulating the production of free radicals by natural immune cells. Motility of the artificial immune cell is facilitated by magnetotactic and other means, as some or all of the constituent particles possess magnetic, especially superparamagnetic properties which may be provided by the same catalytic components since said particles such as those containing compounds such as magnetite, maghemite, and substituted ferrites are capable of catalyzing Fenton-type reactions. Constituent particles of the artificial immune cell may be coated with agents facilitating specific targeting and binding such as antibodies to target antigenes, they may also interact with the intrinsically present natural immune cells by presenting antigens or antibody fragments. Other constituent particles of the artificial immune cell may be coated with lipid bilayers with sequestered biologically active molecules that are released as the lipid bilayer is destroyed by free radicals or they may contain cavities or internal spaces filled with biologically active molecules and capped or sealed with easily oxidizable materials facilitating the release of such molecules in the presence of aggressive oxidants such, thus emulating the property of granulocytes known as degranulation. A variety of the constituent particle may be a capped hollow cylinder with the internal walls presenting with excess of negative electric charges and filled with compacted nucleic acid-protein mixtures whereas upon destruction of the caps the repulsive electric force pushes out the nucleic acid-protein mixture emulating the property of neutrophils known as formation of neutrophil extracellular traps. Modification, modulation and termination of the activity of the artificial immune cell is accomplished by removal by magnetotactic locomotion, disruption with energetic waves, extinguishing of Fenton and Fenton-type reactions by introduction of reaction termination conditions such as excess of antioxidants. Additional refinements are disclosed with specific chemical and physical alterations of the constituent particles or the complete artificial immune cell.

### CROSS REFERENCE TO RELATED APPLICATIONS

This application claims the benefit of U.S. Provisional Application No. 62/436,450 filed on Dec. 20, 2016 to which priority is claimed under 35 U.S.C. 119 and wherein the contents of such are hereby expressly incorporated by reference herein.

### TECHNICAL FIELD

The present invention relates to the techniques of controlled generation of biologically active molecules and specific delivery of said molecules to biologic targets such as cells, tissues, and organs with the disclosed artificial immune cell construct capable of emulating the intrinsic properties of the natural immune cells known as granulocytes including such properties as controlled generation, targeted delivery, passive and active motility, release of previously sequestered chemicals in a controlled fashion and termination of activity based on changes in physical and chemical conditions.

### BACKGROUND

Mammalian and specifically human immune cells include a heterogeneous class known as granulocytes. Typically the class includes such members as neutrophils, eosinophils, basophils and mast cells with certain common properties, some of those being:
ability to synthesize biologically active factors on demand;
presence of active and passive locomotion (motility); ability to release previously sequestered biologically active molecules (degranulation); ability to recognize, bind and interact with specific antigens and other immune cells (adaptive immune competence).

The goal of creating an artificial immune cell with at least the aforementioned properties is hefty but not unreachable. Such artificial immune cell does not have to be a replica or a simplified replica of the natural immune cells but rather may be a plurality of simpler parts such as microscale or nanoscale particles each presenting with one or several of the desired properties and functions, some to a limited extent, some being reasonably close to the properties of the natural immune cells.

The artificial immune cell is unlikely to be capable of complex chemical synthesis of biologically active factors; however, utilization of the well-established chemistry such as Fenton reaction and, more generally, Fenton-type reactions (including the Haber-Weiss reaction) will allow for catalytic generation of a sizable number of free radicals and reactive species of oxygen as well as other reactive species. Constituent particles of the artificial immune cell may supply pro-oxidants and antioxidants consumed in the reaction as well as the catalytic components and sites such as cores and coatings containing compounds of transition and post-transition metals. Natural granulocytes actively produce reactive oxygen and nitrogen species as well as other free radicals utilized as toxic agents and, in smaller quantities, as communication and immune modulation factors.

The artificial immune cell is unlikely to be able to move using the amoeboid movement characteristic of natural immune cells; however, multiple alternatives exist, including magnetotactic movement in which magnetic particles are propelled by magnetic fields. It is worth noting that some of the ferromagnetic and ferrimagnetic materials are also capable of serving as catalysts in Fenton-type reactions and that superparamagnetic particles have the property of not retaining magnetization upon removal of the magnetic field and thus are unlikely to form aggregates; a property that may or may not be useful in the formation of an artificial immune cell. Other approaches to emulation of motility include utilization of the naturally motile organisms (such as bacteria) and cells (such as cells of the immune system as well as spermatozoa) to which nanoscale particles comprising the whole artificial immune cell or its constituent components may be bound.

The degranulation process, in which the natural immune cells release previously sequestered biologically active molecules and the process of release of the neutrophil extracellular net are more easily emulated by the artificial immune cell as it, or it constituent particles may store such molecules in particles' cavities, spaces and in the coatings from which these molecules will be released upon destruction of the constituent particles or their parts and coatings by the oxidation process carried by the free radicals and reactive species generated in Fenton-type reactions by the same particles that carry the sequestered molecules or by other particles comprising the artificial immune cell.

Adaptive immune competence of the natural immune cells is, once again, highly complex and cannot be easily emulated by the artificial immune cell; however, constituent particles can be coated and decorated with immune molecules such as fragments or whole antibodies, antigens, nucleic acids, complement proteins, opsonins, cytokines, enzymes, inflammatory and anti-inflammatory factors as well as small molecule drugs. While such immune competence may be limited and devoid of the ability of the natural immune cells to respond to a staggering variety of stimuli and antigens, in a targeted situation, such as, for example, a specific infection or a specific type of oncologic pathology, a smaller number of signals, antigens, toxins and features needs to be recognized, for which the limited immune competence of the artificial immune cell may be fully sufficient.

Another side of immune competence is the modulation and termination of the activity of the immune cell. The artificial immune cell may be modulated by changing the parameters of Fenton-type reactivity that is driving most other processes taking place in and around the artificial immune cell. Gradual dissolution of catalytic components and cores, delamination of coatings and their dissolution, exhaustion of pro-oxidants and antioxidants necessary for the reaction and consumed in the course of the reaction, poisoning of the catalyst, chelation of the catalytic atoms, ions and compounds, disruption of the integrity of the artificial immune cell with subsequent removal of the magnetotactic particles by external magnetic fields as well as magnetic traps—all this may be attempted and utilized to emulate the properties of the natural immune cells such as apoptosis as well as inhibition of the immune response.

The chemistry of the metal-catalyzed free radical chain reactions in vivo is not completely characterized in all minute detail (see Koppenol; Redox Report 7 (1), pp. 59-60); however, it, and its mechanisms are sufficiently understood to serve as a foundation for the disclosed invention. These types of reactions were first described in the 1890s by the Cambridge chemist H. J. H. Fenton, who utilized the class-defining form of such reactions in his preparation of the Fenton reagent, a strong oxidative mixture of iron salts and hydrogen peroxide. Separately, a class of closely related processes was described by F. Haber and J. Weiss in 1932 as a related reaction that generates hydroxyl radicals from either hydrogen peroxide or superoxide. For the purposes of this disclosure, all related chemical reactions that utilize transition or post-transition metal compounds, oxides or ions as catalysts to generate highly reactive species (free radicals), will be hereinafter referred to as “Fenton-type”, this denotation inclusive of both “classical” Fenton and all variations thereof, including the “classical” Haber-Weiss reaction as a subset. This classification is accepted in biochemical literature (see Nies and Silver, eds. Molecular Biology of Heavy Metals. Springer, 2007, pp. 80-82).

Using the conventions for the Fenton-type chemistry, the first reaction in the cycle is represented as:

Me^{R}+Ox→Me^{Ox}+ROS (Reaction 1)

in which Me^{R} is the reduced form of a transition metal compound, Ox is the oxidizing compound (oxidant), Me^{Ox} is the oxidized form of a transition metal compound, and ROS is the different oxidant, such as a free radical or reactive species derived from Ox in the reaction. A commonly cited example of Reaction 1 is the following classical Fenton reaction:

Fe²⁺+H₂O₂→Fe³⁺+OH⁻+.OH (Reaction 1A)

in which a highly reactive radical .OH is produced from hydrogen peroxide.

The second reaction of the cycle is variable and may be represented as:

Me^{Ox}+AOX^{R}→Me^{R}+AOx^{Ox} (Reaction 2)

in which a reduced form of an antioxidant AOX^{R} reduces the Me^{Ox} compound to Me^{R} compound giving as the second product an oxidized form of an antioxidant AOX^{Ox}. In the specific case of “classical” Haber-Weiss, the antioxidant is the superoxide radical, .O₂—, which, in this narrow and specific case, serves to reduce the oxidized form of the metal compound.

Of essence to this invention is the fact that no matter how these reactions are termed or named, they all share very specific feature, such as that the metal compounds are not consumed in the chain reaction; rather these compounds serve as catalysts in the production of the highly reactive species also known as free radicals.

As these reactions are very close in mechanisms, they are commonly termed “Fenton-type” without the implication that only the “classical” Fenton or “classical” Haber-Weiss reactions are employed in various embodiments of the disclosed invention.

In place of ions of transition metals, oxides of such metals or metalorganic chemicals can be utilized to serve as catalysts in the aforementioned Fenton-type reactions. As reported in literature (Wardman and Candelas; Radiat. Res., 145, pp. 523-531), the first part of the cycle is then the oxidation of the transition metal compound to a higher oxidation state, for example, iron (II, III) oxide, Fe₃O₄ may be oxidized to iron (III) oxide, Fe₂O₃. In this, oxidant is consumed, such as molecular oxygen, hydrogen peroxide, superoxide radical or other reactive species. The second part of the cycle, which allows it to proceed in a cyclical fashion, is the regeneration of the transition metal compound to its lower oxidation state, such as, for example, iron (III) oxide, Fe₂O₃ is reduced to iron (II) oxide, FeO or back to iron (II, III) oxide, Fe₃O₄, both of which are capable of entering the first reaction of the cycle. In this, the second part of the reaction, antioxidant is consumed. Free radicals may be produced in the first, the second reaction, or in both, depending on the specific compounds.

If, for example, ascorbic acid (Asc), chosen here for its ubiquity in living organisms, is utilized as the antioxidant in Reaction 2, the possible chemical reactions are:

Fe³⁺+AscH⁻→Fe²⁺+.Asc⁻+H⁺ (Reaction 2A)

Fe³⁺+Asc²⁻→Fe²⁺+.Asc⁻ (Reaction 2B)

This chemistry is known since the 1970s and 1980s, with the realization that the presence of significant amounts of ascorbate dramatically increases toxicity in diseases associated with iron overload (such as hemochromatosis), which is attributed to the overproduction of free radicals (Burkitt and Gilbert, Free Rad Res Comm 10(4-5), pp. 265-280) while the ascorbate radical is stable and the observed toxicity cannot be attributed to it.

It is also known that aerobic processes in living cells readily produce partially reduced forms of molecular oxygen (O₂), commonly termed as reactive oxygen species (ROS), inclusive of H₂O₂ (hydrogen peroxide) and .O₂ (superoxide radical). Accepted view is that in the presence of ions or oxides of transition metals (such as Fe and Cu) that serve as catalysts, the process is driven by the Fenton-type chain reactions (Lemire et al.; Nature Reviews Microbiology, 11, pp. 317-384).

Free radicals, such as reactive oxygen species (ROS), together with similarly reactive species of nitrogen and halogens, as well as radicals of organic molecules (e.g., the tyrosine radical) are known to have multiple effects on the biological tissue, including the ability to induce apoptosis (programmed cell death) in cancerous tissues, promote wound healing and destroy/deactivate infectious agents such as bacteria, viruses, protozoa and fungal cells. While the generalized production of free radicals in a living organism is commonly viewed as toxic, a more localized production is one of the natural features of the activity of the immune system as well as many other biochemical processes. Underproduction of ROS is known, inter alii, weakened defense against microorganisms including such common pathogens as Salmonella enterica, Staphylococcus aureus, Serratia marcescens, and Aspergillus spp.

It is known that granulocytes as well as other immune cells produce potent streams of free radicals, especially during the degranulation response. The disclosed invention allows for an embodiment that is similar in its properties to such behavior of immune cells and specifically granulocytes as it allows for targeted and controlled production of free radicals and release of other chemicals from the microscopic particles prepared according to the disclosed method and specifications.

Another principle relevant and essential to the disclosed invention is that of motility as the property of magnetotactic particles, characterized by their ability to be attracted to external magnetic fields without becoming magnetized and, thus, without forming aggregates in the absence of an external magnetic field. These are well known in the art and the relevant property of some of the multiple varieties of such particles is known as superparamagnetism, which is present when the size of the particle of a ferromagnetic or ferrimagnetic material [such as iron (II, III) oxide, Fe₃O₄ (magnetite) or gamma-iron (III) oxide, Fe₂O₃ (maghemite, rust)] is small enough to be of the size of the single magnetic domain (less than approximately 128 nm in diameter for near-spherical particles of magnetite) or smaller.

Such superparamagnetic particles are attracted by magnetic fields but do not form aggregates in the absence of a magnetic field, allowing them to be delivered as fine suspensions in non-magnetic media, such as water- or lipid-based solutions. It is of essence to the disclosed method that many of the compounds exhibiting the desirable magnetotactic properties are also capable of serving as catalysts in Fenton-type reactions.

The use of magnetotactic particles for localization may require the biological tissue in which the particles are localized to be submitted to magnetic flux densities in excess of 1 tesla; however, decades of use of strong magnetic fields in nuclear magnetic resonance (magnetic resonance imaging (MRI) technology routinely utilizes densities from 0.2 to 7.0 tesla) proves the lack of toxicity and relative safety of such strong magnetic fields and, thus, applicability for the disclosed use. Alternatively, permanent magnets may be implanted into the target tissue, such as a solid tumor, smaller magnetic particles can be applied topically onto a wound, insufflated, infused in the form of a suspension, forced through the tissues with strong magnetic fields, sonic energy (e.g., ultrasound waves), or through utilization of electric potential gradients, similar to iontophoresis or electro-osmosis.

The idea of using magnetotactic particles in medicine is also well known, with numerous examples ranging from the use of these particles as contrast media for magnetic resonance imaging, to the use of such particles as vehicles of targeted delivery of chemotherapy drugs to cancer cells. The U.S. Pat. No. 8,088,358 B2 issued on Jan. 3, 2012 to M. Haasse et al. teaches the preparation of paramagnetic nanoparticles and their use as nuclear magnetic resonance enhancers; however, this and other issued patents do not claim the utilization of the Fenton-type (or the closely related Haber-Weiss-type) reactions to generate free radicals in a targeted and controllable fashion, nor do these patents teach the methods that allow for utilization of microscopic particles to mimic the properties and activities of granulocytes and other immune cells.

Multiple patents had been issued in the field of nanotechnology, including patents that utilize nanoparticles as scavengers of free radicals; however, to our knowledge no patent had been issued for microscopic particles that are utilized for targeted generation of free radicals in biological tissues by utilizing Fenton-type reactions.

The U.S. Pat. No. 9,259,468 B2 issued on Feb. 16, 2016 to A. Gesguiere et al. teaches the method and preparation of conjugated polymer nanoparticles that create reactive oxygen species (ROS) upon activation with light or other means; these particles do not incorporate either the catalysts or the substrates of Fenton-type or any related chemical reactions and the inventors do not claim any other means of activation except activation with light of high intensity, at least 60 J/cm² (claim 1) or 120 J/cm² (claims 4 and 5) for an unspecified amount of time. The aforementioned invention substantially and wholly differs from the disclosed invention that does not rely on activation by light or any other physical source of energy.

Another example of prior art is the U.S. Pat. No. 8,651,113 B2 “Magnetically responsive nanoparticle therapeutic constructs and methods of making and using” in which particles of magnetite (Fe₃O₄) with sizes of 1-50 nm and likely exhibiting superparamagnetism are utilized for targeted delivery of compounds such as chemotherapy drugs using directional variable magnetic fields leading to accumulation in the target tissue with subsequent extravasation and endocytosis.

The most important differences between the disclosed invention and the granted US Patent include: the aim of the granted US Patent is to develop a system of delivery to target tissues, while the goal of the disclosed invention is to create a system that emulates certain vital and characteristic properties of the natural immune cells and most importantly possess the ability to respond to the changes in their milieu. To accomplish this goal, the disclosed invention utilizes Fenton-type reactions to produce free radicals or reactive species that are further used as toxic agents or further serve to initiate and sustain desirable chemical reactions; the use of assemblies of particles of different kinds such as coated with different materials that supply consumable compounds and sustain production of free radicals or reactive species with said aggregates presenting with reactivity and responsivity to the changes in the targeted structures. Said Fenton-type reactions are facilitated by catalysts that, in some embodiments, possess the magnetotactic properties such as superparamagnetism that is also utilized in the granted US Patent; however, the granted patent is limited to the use of iron oxides (such as magnetite) while the disclosed invention utilizes both iron oxides and other magnetic materials such as preparations of pure elements such as cobalt, nickel, copper, ruthenium, gold and other transition and post-transition metals, their alloys and chemical compounds. Most importantly, the granted Patent does not aim to emulate properties of the natural immune cells as its aim is a delivery system and particles of potentially reactive magnetite are intentionally coated to render them inert.

### SUMMARY OF THE INVENTION

The disclosed invention is of an artificial immune cell, that is, a construct or an aggregate of constituent particles which, in turn, may be comprised of specific components, that is capable of emulation of the specific properties of natural immune cells, specifically granulocytes with the said properties being:
generation of biologically active factors;
presence of active and passive locomotion (motility);
ability to release previously sequestered biologically active molecules (degranulation);
ability to recognize, bind and interact with specific antigens and other immune cells (adaptive immune competence).

The first emulated property, that of generation of biologically active factors, is attained by incorporation into the artificial immune cell constituent particles that utilize Fenton-type chemical reactions to produce free radicals or other reactive species of oxygen and other highly reactive chemicals, with ions, oxides and other compounds of transition and post-transition metals serving as catalysts and consumable chemicals including pro-oxidants and antioxidants either supplied by the biochemical milieu of said artificial immune cell or are incorporated into the constituent particles of the artificial immune cell.

The second emulated property, that of motility of the artificial immune cell is attained by incorporation into the artificial immune cell of magnetotactic particles, including superparamagnetic cores and coatings of said particles that are propelled by external magnetic fields and other similar methods as a preferred embodiment, or alternately, attachment of said particles to naturally motile organisms such as bacteria or naturally motile cells such as immune cells intrinsically present in the milieu of the artificial immune cell or spermatozoa and similar actively propelled cells.

The third emulated property, that of degranulation of natural immune cells such as granulocytes, is attained in the artificial immune cell by incorporation of constituent particles that contain sequestered biologically active factors either inside the cores of said particles or within the coatings of said particles or both. As sequestering components of said particles are degraded by the action of free radicals and other reactive species, the sequestered biologically active factors are released.

The fourth emulated property, that of adaptive immune competence is attained by incorporation of immune system-derived and other biochemically active molecules and other factors to mitigate, modulate, enhance and terminate the activity and integrity of the artificial immune cell; preferred examples being the use of molecules such as antibodies and other immune factors as well as small molecule drugs to facilitate selective and specific recognition of antigens, microorganisms, cells, tissues, organs and extracellular and intracellular components with subsequent induction of change of the parameters of Fenton-type reactivity, engagement with natural immune cells and to achieve termination of the activity of the artificial immune cell, controlled disruption of the integrity of the artificial immune cell with subsequent removal of the constituent particles and other similar means.

The disclosed invention does not address methods of manufacture of said particles, nor specifics of attainment of motility via magnetotactic means such as the use of pulsed, moving and disrupted magnetic fields, or other means, as such methods are well-established in the art, but presents a variety of properties and compositions of cores, coatings, decorations and components of said particles together with the preferred methods of combining and utilizing of said particles to achieve sufficient emulation of the properties of the natural immune cells.

### BRIEF DESCRIPTION OF THE DRAWINGS

In order to illustrate the manner in which the above-recited and other advantages and objects of the invention are obtained, a more particular description of the invention briefly described above will be rendered by reference to several specific embodiments thereof, which are illustrated in the appended drawings with the understanding that these drawings depict only typical embodiments of the invention and are not therefore to be considered limiting in scope. The invention will be described and explained with additional specific detail through the use of the accompanying drawings.

FIGS. 1, 2, 3 and 4 is a schematic illustration of the chemical reactions referred to as Fenton-type in this disclosure.

FIGS. 5, 6, 7, 8, and 9 present general schematic depiction of the different varieties of the microscopic particles encompassing various embodiments that upon assembly or aggregation constitute the disclosed artificial immune cell.

FIGS. 10, 11, 12, and 13 depict the hollow core particle and its reactivity, with said particle being a constituent particle of the disclosed artificial immune cell designed to facilitate emulation of the property of natural immune cells known as degranulation.

FIGS. 14, 15, 16, 17, 18, and 19 depict the alternate embodiment of a constituent particle of the artificial immune cell, this time utilizing molecular bilayers to sequester and subsequently release biologically active molecules and factors in a manner that emulates the property of natural immune cells known as degranulation.

FIGS. 20, 21, and 22 illustrate another optional embodiment of a constituent particle of the artificial immune cell, this time utilizing a hollow cylinder core capped with closure structures that are degraded by oxidative insult leading to the release of previously sequestered granules of a nucleic acid-protein mixture that unfolds upon release forming a web-like structure, thus allowing the artificial immune cell to emulate the property of the natural immune cells, namely neutrophils, to emit the structure known as the neutrophil extracellular net.

FIGS. 23, 24, and 25 illustrate the concept of the artificial immune cell as a construct (FIG. 23) or an aggregate (FIG. 24) of the constituent particles; while FIG. 25 depicts the attack of both types of the artificial immune cell on cancerous cells with motility provided by external magnetic fields.

### DETAILED DESCRIPTION OF THE INVENTION

The disclosed artificial immune cell is either a construct or an aggregate of constituent particles whereas a construct is a plurality of constituent particles assembled into a construct held together by chemical bonds, while an aggregate is a plurality of constituent particles not linked by chemical bonds but held together by other means, such as hydrophobic interactions, electrostatic forces, magnetic forces, rotational forces or other similar means that exclude chemical bonding. In both cases the constituent particles may be simple, i.e., consisting of one component or complex, i.e., consisting of several components such as cores, coatings, caps and seals if the core contains void spaces, cavities and similar features, can be decorated with attached functional groups and molecules and the such.

In one embodiment such constituent particles contain a core with or without coatings and decorations with such core suited for the purposes of catalysis of Fenton-type reactions.

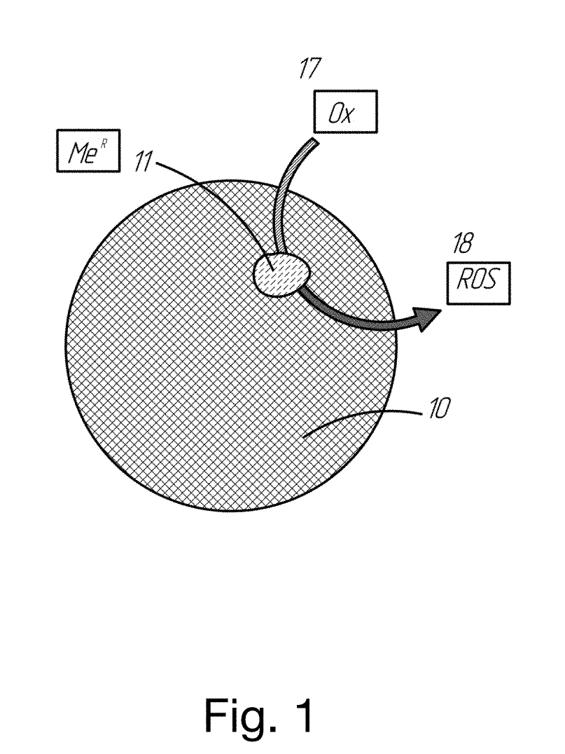

FIGS. 1, 2, 3, and 4 depict the steps of Fenton-type reactions as catalyzed by the core 10 of such constituent particle in which free radicals and other reactive species are produced, while initial oxidants are consumed with subsequent regeneration of the catalytic site by the molecules of the antioxidant that is also consumed in the reaction.

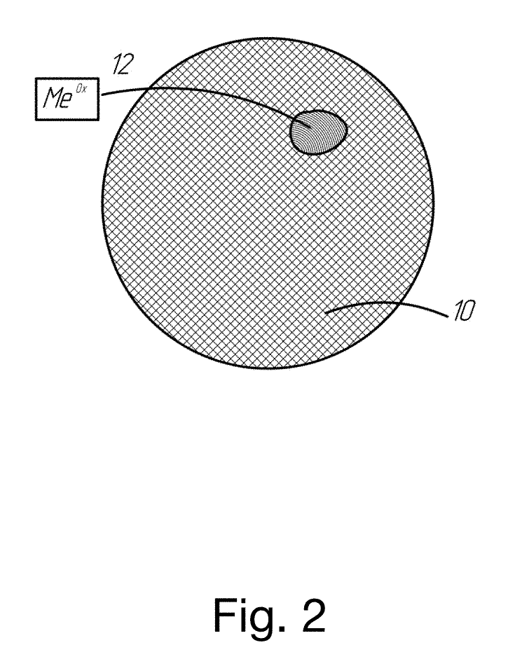

FIGS. 1 and 2 illustrate aforementioned Reaction 1 whereas as depicted in FIG. 1 the molecule 17 of the initial oxidant Ox attacks the catalytic site 11 such as ion(s), oxide(s), other compound(s) the transitional or post-transitional metals present on the surface of the core 10 of the constituent particle of the disclosed artificial immune cell. Upon such attack, oxidant 17 is transformed into molecule 18 which is a representative of reactive species ROS, in this case, reactive species of oxygen. The composition of the catalytic site 11 also changes as depicted in FIG. 2, whereas the reduced form of the metal ion, oxide, other compound of the transitional or post-transitional metal MeR is transformed into the oxidized form of the same ion, oxide, other compound of the transitional or post-transitional metal 12.

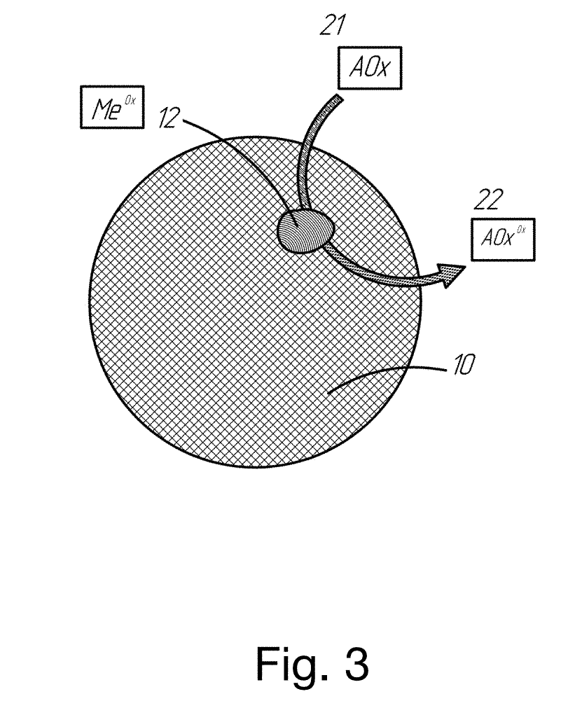

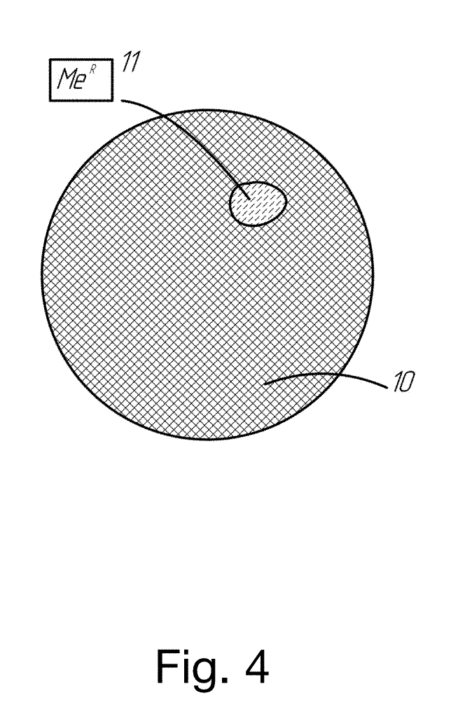

FIGS. 3 and 4 illustrate aforementioned Reaction 2 whereas as depicted in FIG. 3 the molecule 21 of an antioxidant AOx attacks the oxidized catalytic site 12 and reduces the oxidized ion(s), oxide(s) or other compounds of the transitional or post-transitional metals MeOx, in the process, the oxidized form 22 of the antioxidant AOxOx is produced. FIG. 4 depicts the regenerated catalytic site 11 that with the reduced form 11 of the ion(s), oxide(s) and other compounds of the transitional or post-transitional metals MeR is again available to perform catalysis of Fenton-type reactions.

The chemical identity of the catalytic site is hereby disclosed as an integral part of the disclosed invention. Fenton-type reactions may be catalyzed by ions, oxides and other compounds of the transitional or post-transitional metals as depicted, with said metals belonging to of Groups 3 (IIIB) through 12 (IIB) of the Periodic table. Compounds of said elements suitable for the purposes of catalysis of Fenton-type reactions include oxides, salts, metalorganic compounds or complexes of transition and post-transition metals, with iron oxides being the most preferred choice, but other compounds, such as copper oxides being highly usable, other iron and copper compounds being desirably usable and nickel, chromium, vanadium, titanium and cobalt compounds also potentially usable, as are all ions, oxides, compounds and complexes of other transitional and post-transitional metals, notably including ruthenium and gold.

Alternately, Fenton-type reactions may be catalyzed by substituted ferrites with copper taking the place of some of the iron atoms, layered alumosilicates and carbon nanotubes, as well as gold nanoparticles with or without incorporated other elements, as well as platinum group elements, notably ruthenium, in the form of nanoparticles or chemical compounds.

Said catalytic sites may be also be prepared from non-metallic particles such as appropriately folded and shaped biological compounds, such as nucleic acids or prepared from natural microscopic particles, such lipoproteins (chylomicrons, VLDL, LDL, IDL and HDL particles, commonly known as blood lipids) which are coated or loaded with aforementioned catalytic atoms, ions, oxides and other compounds or nanoparticles of transition and post-transition metals.

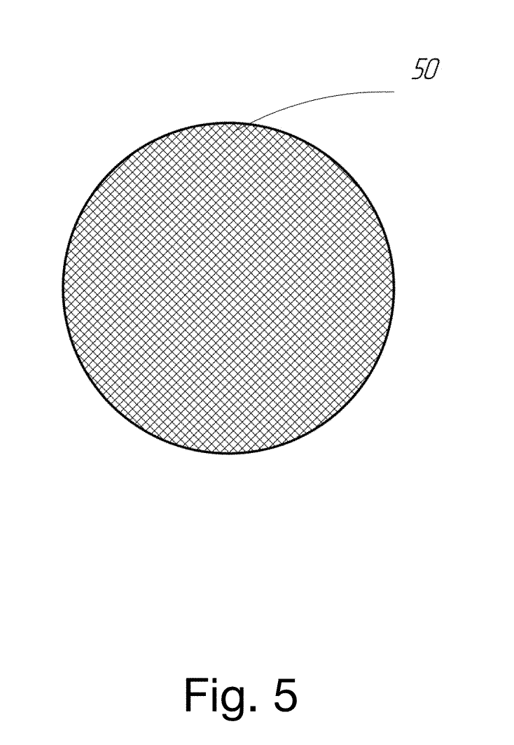

FIGS. 5, 6, 7, 8, and 9 depict further refinements of the constituent particles from which the artificial immune cell is constructed or aggregated. Not all possible combinations of cores and coatings are depicted with the understanding that the depicted combinations are to be considered as illustrative and not restrictive.

As noted above, the constituent particles may also be of simple composition consisting of an isolated non-coated core or of complex composition containing two or more constituent components such as a core and a coating with additional decorations attached or incorporated into the coating.

Cores of the particles may be selected for the possession or lack thereof of the magnetotactic property facilitating the movement of the core and its associated coatings (if present) in magnetic fields. FIG. 5 depicts the simple magnetotactic core 50 depicted as a spherical particle without any coatings. Such core may be manufactured from a material that combines two properties: being magnetotactic and being able to serve as a catalyst for Fenton-type reactions. Examples of such materials include certain oxides of iron, such as magnetite (Fe₃O₄) and maghemite (gamma-Fe₂O₃) with multiple other embodiments possible as described below.

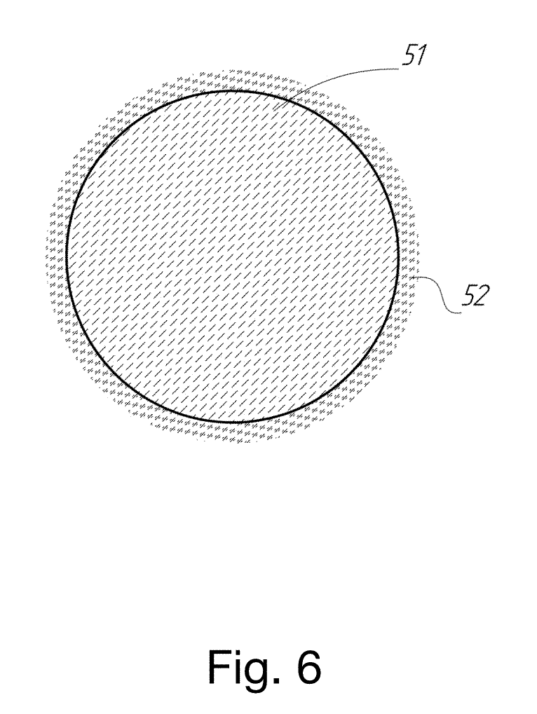

FIG. 6 depicts a constituent particle of the artificial immune cell construct or aggregate which is a complex particle consisting of two components, core 51 and coating 52. Core 51 as depicted is magnetotactic, however, it does not contain atoms, ions, or compounds capable of catalyzing Fenton-type reactions. An example of such magnetotactic core material is gadolinium, an element that possesses ferromagnetic properties but by itself and in various compounds does not serve as catalyst of Fenton-type reactions and may even serve as inhibitor or catalytic poison of Fenton-type reactions. Such property as inhibition of Fenton-type reactions is advantageous in emulation of properties of natural immune cells that produce free radicals and reactive species in an intermittent, rather than continuous fashion, as well as in termination of the activity of the artificial immune cell by incorporation or aggregation of inhibiting particles into or onto the artificial immune cell.

To further inhibit or block the Fenton-type reaction, the depicted core 51 of the particle is coated with a layer 52 of an inhibitory compound, such as a chelating substance capable of chelating the catalytic atoms, ions and compounds of iron, copper and other transition and post-transition metals. An example of such chelating substance is deferoxamine, an iron-specific chelator.

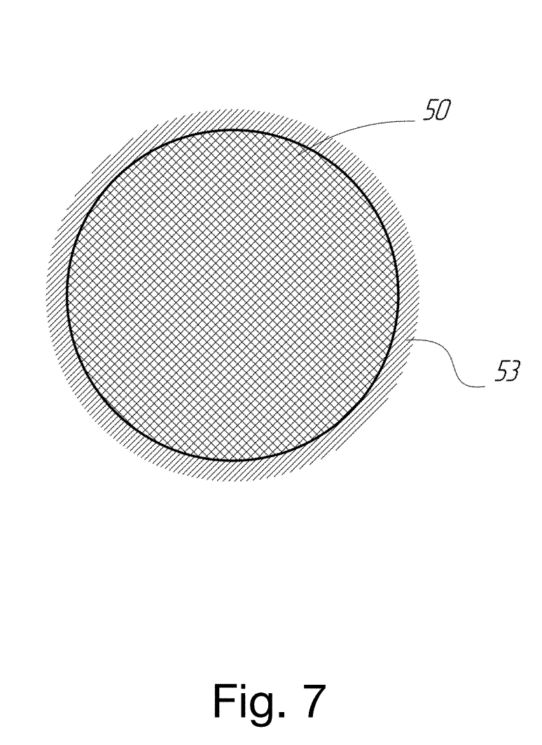

FIG. 7 depicts a variation of the constituent particle of the artificial immune cell that is complex and is designed to enhance the course of Fenton-type reactions. It consists of a magnetotactic core 50 made from a material that also functions as a catalyst for Fenton-type reactions and is coated with a layer 53 of an antioxidant compound or mixture of compounds. One example of said material 50 is aforementioned magnetite (Fe₃O₄) and one example of said antioxidant compound 53 is calcium ascorbate, a salt of ascorbic acid (vitamin C).

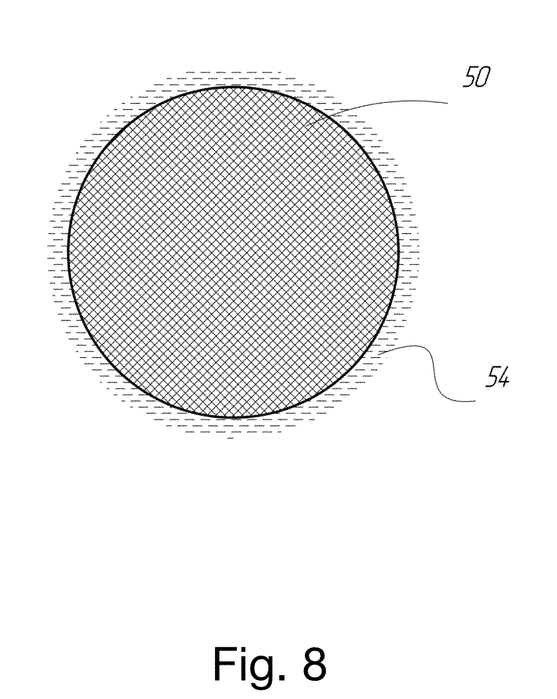

FIG. 8 depicts another variation of the constituent particle of the artificial immune cell that is complex and is designed to enhance the course of Fenton-type reactions. It consists of a magnetotactic core 50 made from a material that also functions as a catalyst for Fenton-type reactions and is coated with a layer 54 of pro-oxidant compound or mixture of compounds. One example of said material 50 is aforementioned magnetite (Fe₃O₄) and one example of said pro-oxidant compound 54 is sodium perborate Na₂H₄B₂O₈, a compound that upon hydrolysis produces hydrogen peroxide H₂O₂ as well as perborate anion (B(OOH)(OH)₃⁻), both being suitable as oxidants 17 in Fenton-type reactions.

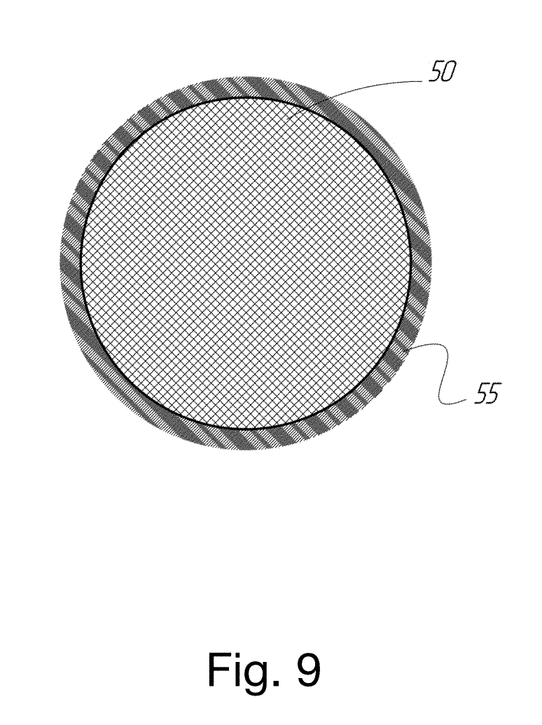

FIG. 9 depicts another variation of the constituent particle of the artificial immune cell that is complex and is designed to facilitate emulation of such properties of the natural immune cells as ability to recognize, bind and interact with specific antigens and other immune cells (adaptive immune competence). The particle is depicted as containing a magnetotactic core 50 that is coated with a layer 55 of biologically active molecules that facilitate recognition, binding and interaction of the particles with antigens and natural or other artificial immune cells. One example of said material 50 is the aforementioned magnetite (Fe₃O₄) and one example of said biologically active molecules is immunoglobulin antibody with desirable specificity, such as, e.g., against melanoma-associated antigen (MAGE) allowing recognition and binding to said protein with subsequent release of free radicals and other reactive species in the immediate vicinity of this tumor antigen potentially destroying the melanoma cells that produce this antigen.

FIGS. 5, 6, 7, 8 and 9 are drawn without indication of the scale and size of the depicted particles; they also present a simplified depiction of the shape of the particles which necessitates the following essential disclosures:

Sizes of the constituent particles of the artificial immune cell may be either on microscale or nanoscale. The microscale particles are characterized by their diameter or the length of the longest side measured from single to hundreds of micrometers and the nanoscale particles are characterized by their diameter or the length of the longest side measured from fractions of a nanometer to hundreds of nanometers. The guiding biological consideration in choosing the size of the particles for the specific embodiment of an artificial immune cell is: microscale particles are utilized in the cases when obturation of blood and lymph vessels and extracellular spaces is not to be avoided and the processes of extravasation and endocytosis are not essential or outright undesirable and the consideration that nanoscale particles are utilized in the cases when obturation of blood and lymph vessels and extracellular spaces is to be avoided and the processes of extravasation and endocytosis are essential or desirable.

Additional consideration in regard of the size of the particle cores that possess the magnetotactic property is the desire to employ the phenomenon of superparamagnetism whereas nanoscale particles of ferromagnetic and ferrimagnetic materials such as aforementioned magnetite and maghemite smaller or approximately equal to the size of the single magnetic domain of the material possess the property of being attracted by magnetic fields but in the absence of an external magnetic field their magnetization averages to zero. The maximum desirable size of particles of magnetite to exhibit superparamagnetism is 29-36 nm, while other materials may present with superparamagnetism in smaller or larger particles.

The shapes of constituent particles of an artificial immune cell may be spherical or near-spherical as depicted in FIGS. 5-9 or cylindrical, cubic, that of a parallelepiped as well as irregular, with some of the varieties of said particles possessing internal voided volumes such as cavities and hollows, such as hollow spheres, hollow cylinders and the such. The hollows may be connected with the outside of the particle forming an aperture which may be capped, sealed or closed by coatings or distinct seal, trap and cover-like structures attached to the coating or the core of the particle.

Additional considerations regarding the choices for molecules that may be incorporated into cores 50 of constituent particles of the disclosed artificial immune cell include such materials as iron oxides (magnetite, Fe₃O₄ and maghemite, gamma-Fe₂O₃), mixed ferrites containing iron, oxygen and other elements such as copper, cobalt, nickel, ruthenium, gold and other transition and post-transition metals and their chemical compounds. Generally, compounds of Fe and Cu may be preferred due to better biological compatibility, except when such is not desired, whereas compounds of Ni, V, Co, Cr, Ti, Ga, In and other less biologically compatible compounds are preferred, e.g., in treatment of oncological conditions.

Additional considerations regarding the choices for molecules that may be incorporated into coatings include:

For coatings 53 containing antioxidant chemicals compounds such as ascorbic acid (vitamin C) and its salts and esters, species of tocopherols and tocotrienols (vitamin E), reduced forms of cofactors such as nicotinamide adenine dinucleotide (NADH), nicotinamide adenine dinucleotide phosphate (NADPH), flavin adenine dinucleotide (semiquinone FADH and hydroquinone FADH2), uric acid, ubiquinol (coenzyme Q), alpha lipoic acid, glutathione, carotenoids and retinoids, polyphenols such as resveratrol, flavans and flavonoids, as well as substantially similar compounds may be considered with the primary choice of ascorbic acid and its derivatives for particles designed to function in environments with high partial pressure of oxygen;

For coatings 54 containing oxidants or pro-oxidants chemicals and compounds such as hydrogen peroxide, lipid peroxides, organic peroxides including benzoyl peroxide and carbamide peroxide, inorganic peroxides including sodium perborate and sodium percarbonate, elemental or molecular oxygen, singlet oxygen, ozone, radicals of oxygen such as superoxide radical, radicals of nitrogen such as nitric oxide or peroxynitrite, compounds of halogens such as hypochlorous acid and the like, exogenous pro-oxidants such as menadione (vitamin K3, C₁₁H₈O₂), antibiotics such as doxorubicin (C₂₇H₂₉NO₁₁) and bleomycin (C₅₅H₈₄N₁₇O₂₁S₃), toxins inclusive of paraquat (C₁₂H₁₄Cl₂N₂), and potassium triiodide (I₂KI) as well as other suitable compounds capable of entering into Fenton-type reactions, including complex systems relying on endogenous enzymes such as NADPH oxidase, xanthine oxidase, myeloperoxidase and monoamine oxidase that generate free radicals in the presence of molecular oxygen or other oxidant substance, with additional consideration given to solutions of oxygen and singlet oxygen in solids and liquids including but not limited to fluorocarbon compounds such as octadecafluorodecalin (perfluorodecalin) and the like as well as chemically bound ground state triplet oxygen and singlet oxygen such as to hemoglobin, myoglobin, hemocyanin and similar molecules. Additionally, oxygen diffusion-enhancing compounds, such as crocetin and its derivatives (carotenoids), to increase oxygenation of the environment in which the artificial immune cell is to function. Additionally, pro-oxidant sensitizers including radio-sensitizers, misonidazole, etanidazole (functioning as glutathione suppressor and inhibitor of glutathione S-transferase), metronidazole, and tirapazamine that under low oxygen conditions produces *OH radical, especially under reductive conditions as well as 12-O-Tetradecanoylphorbol-13-acetate as an inducer of endogenous superoxide production.

Reduced speed of solubilization of coatings 53 and 54 may be achieved by incorporation into the coating or supracoating with neuraminic acid, sialic acids or similar saccharides to delay the production of free radicals and reactive species; also mixtures of substrate-enzyme may be utilized to accomplish the same such as polysialic acids with neuraminidase, bromelain or other proteolytic enzyme with gelatin or other digestible protein, nucleic acids with deoxyribonuclease or ribonuclease and the like.

Additional considerations in choosing the compounds to be incorporated into coatings 55 containing biologically active molecules include the use of antibodies capable of recognizing and binding to specific antigens, fragments of complement proteins such as C1q, C4b and C3b that recognize and bind the Fc portion of an antibody molecule when bound to an antigen, as well as strong binding observed in the biotin-streptavidin and biotin-avidin systems. In some embodiments of the disclosed artificial immune cell, especially those embodiments useful in interaction with cancerous, precancerous and transformed cells the constituent particles, their cores and coatings may incorporate APEX-1 (abasic site repairing enzyme) or other repairing chemicals, hoping to force maturation on the cancer cell following the introduction of oxidative stress.

Said coatings 53, 54, and 55 may also incorporate neutral molecules and structural components such as organic polymers, carbon nanotubes, graphene sheets, other carbon-derived substances, as well as carbohydrate derivatives, such as carboxymethyl cellulose, starches, glycogens and glycoproteins being considered, as well as amino acid polymers, as well as non-encoding nucleic acids, their analogs and derivatives.

Alternately, the coating may be manufactured from lipids, as to mimic the composition of biological cellular membranes and allow adhesion and integration of the particles onto cellular membranes. In this case, phospholipids, glycolipids, and sterols, as well as free unsaturated fatty acids, cholesterol and other similar molecules may be used, forming mono- or bilayers on the reactive core of the particle. The pro-oxidant chemicals that may be carried in the lipid-based coating include, in one implementation, peroxides of fatty acids with other organic radicals included as an option. Antioxidants that may be carried in the lipid-based coating include, in one implementation, vitamin E (tocopherols and tocotrienols), as well as other fat-soluble antioxidants, such as retinoids and carotenoids. Other chemical compounds may be intercalated between the two constituent layers of a lipid bilayer, such as drugs, enzymes, cofactors and other desirable chemicals.

Additionally, two or multiple coatings, undercoatings and supracoatings may be implemented, with a variety of chemical properties, such as the undercoating containing the pro-oxidant chemicals and the supracoating comprised of organic polymers or lipids, possibly including sequestered compounds in pocket- or bubble-like structures formed between layers of coatings. Such coatings may contain compounds that respond, e.g., by changing their structure, conformation or physical and chemical properties under low (acidic) pH conditions associated with inflammation, infection, apoptosis or necrosis of cells, such as observed in lysis of tumors.

The coating 55 may also contain biologically active molecules, which may facilitate vasodilation, vasoconstriction, extravasation, immune activation or inhibition as well as adhesion to specific targets and may protect the unintended targets. In some cases, the coating may incorporate a prodrug (a precursor of a pharmacological remedy) which is converted to the active form of the drug by the free radicals generated by the particles in the Fenton-type reactions.

Additionally, functional coatings designed to facilitate extravasation can be applied on the particles; such coatings may include proteins and other compounds similar to those found on the surface of a metastatic cancer cell.

Additionally, the coating may include other relevant chemicals and their mixtures. In one example, incorporating the amino acid arginine together with a purified preparation of the enzyme nitric oxide synthase and necessary cofactors, allows for generation of nitric oxide either during the initial travel of the particle to the target tissue or when the particle is lodged in the target tissue, as well as both, depending on the amount of arginine and necessary cofactors incorporated in the particle as well as its rate of transit to the target tissue. This accomplishes several goals: generation of nitric oxide, which is a vasodilator and may facilitate better blood flow through the target tissue, allowing for more rapid and more reliable delivery of the particles; and subsequent sequestration of nitric oxide and generation of the biologically active peroxynitrite from nitric oxide and reactive species of oxygen, especially superoxide produced by the Fenton-type reactions of the particle core with pro-oxidants and antioxidants. Peroxynitrite is a reactive species of nitrogen as well as a strong oxidizer and may be relevant in treatment of certain disease conditions more so than the reactive species of oxygen.

In yet another embodiment of the disclosed invention, the coating 55 may include chemicals that are photosensitive, luminescent, phosphorescent or possess other properties related to emission, absorption, and re-emission of light or other electromagnetic radiation, inclusive of gamma rays as produced by the unstable isotopes of transition and post-transition metals.

The choice of specific constituent particles with their specific cores and coatings to form a particularly effective artificial immune cell depends on the specifics of the biological target. For instance, the inner parts of a solid cancerous tumor are known to be strongly hypoxic, thus, the need for particles supplying either oxygen or other oxidants is suggested. At the same time, tumors of the lung tissue, cancerous cells suspended in the bloodstream, as well as damaged tissues in open wounds, and respiratory tissues affected by infections are not generally hypoxic, thus, the constituent particles may be chosen to deliver antioxidant chemicals. Of note is the fact that some chemicals traditionally viewed as antioxidants may become pro-oxidants under the conditions observed in respiratory tissue, an example of such is beta-carotene that can, therefore, be utilized as a “mutable” compound, suitable for either the antioxidant or the pro-oxidant species of the localizable particle.

Motility of the artificial immune cell may be achieved by the aforementioned magnetotactic phenomena; however, it may be produced in select embodiments by attachment of the constituent particles to intrinsically motile biologic objects such as motile bacteria, immune cells, and spermatozoa whereas such motile biologic objects propel themselves and the attached particles utilizing the activity of flagella, pseudopodia, cytoskeletal filaments, cilia and pili to produce directional movement guided by the presence and gradients of electromagnetic fields, gradients of chemical nature such as gradients of pH, ionic and non-ionic compounds serving as biochemical attractors and other means of directing and controlling the intrinsic motility of such biologic objects.

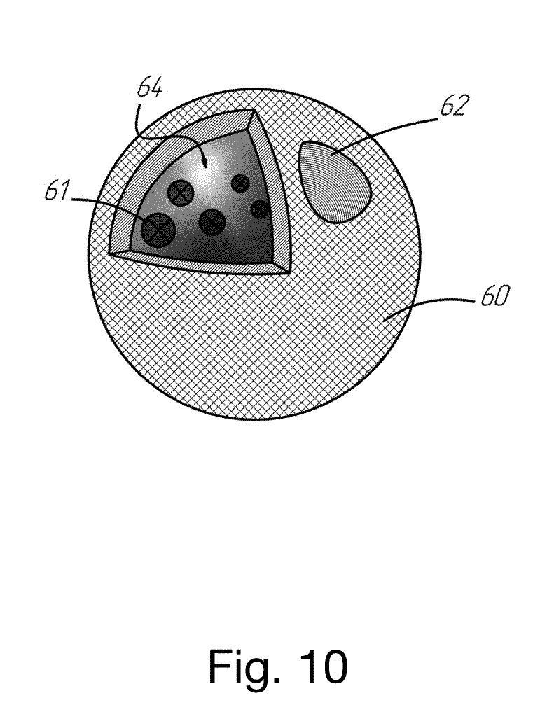

FIGS. 10, 11, 12, and 13 depict one of the possible embodiments of a constituent particle of the artificial immune cell designed to facilitate the release of previously sequestered molecules or factors from a hollow particle thus emulating the property of the natural immune cells such as granulocytes known as degranulation.

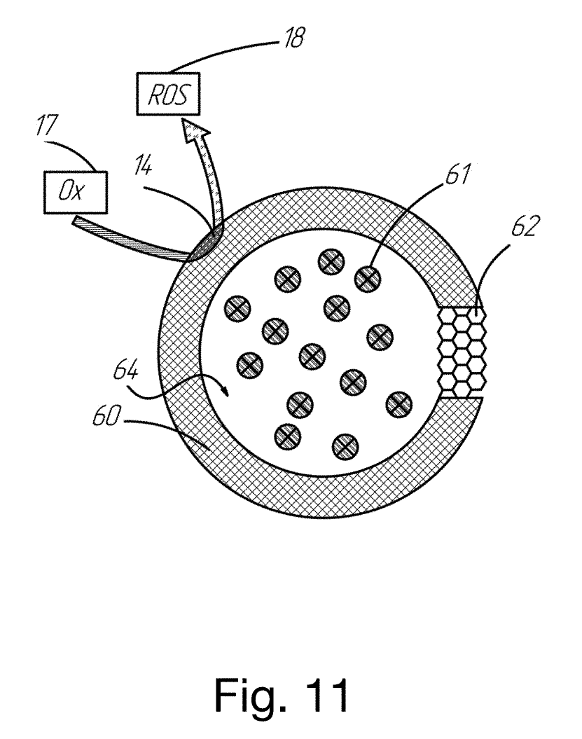

FIG. 10 presents the schematic drawn in pseudo-3D of said embodiment of a constituent particle of the artificial immune cell in which the constituent particle possesses a hollow magnetotactic and catalytic core 60 with cavity 64 containing sequestered molecules or factors 61 and with an closure, seal or trap 62 applied onto the aperture that connects the cavity 64 with the outside of the particle. FIG. 11 presents the cross-section of said particle with the magnetotactic and catalytic core 60 revealing the internal cavity 64 filled with sequestered molecules or factors 61 and with aperture closed by the closure, seal or trap 62. Of note is the presence of the catalytic activity facilitating generation of free radicals or reactive species 18 from the initial oxidant 17 on the catalytic site 14.

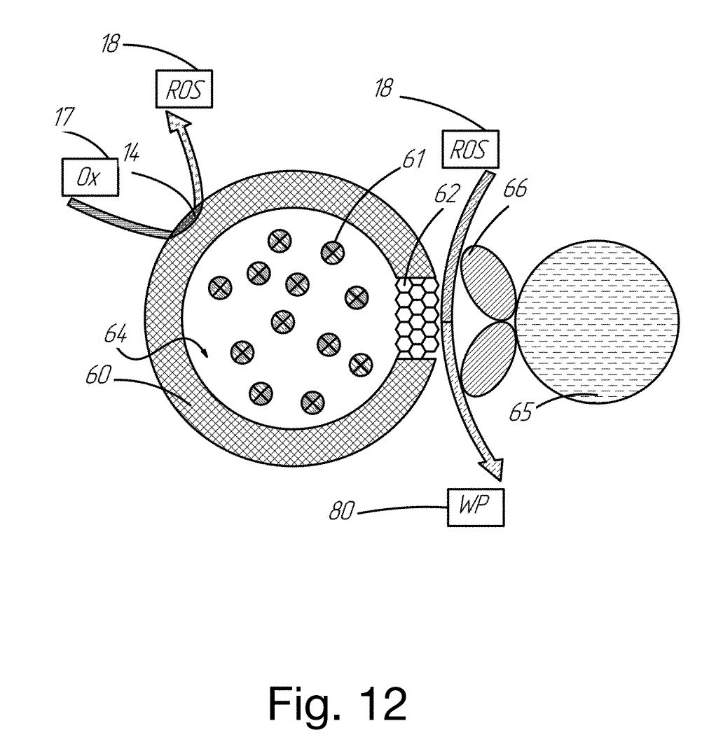

FIG. 12 illustrates the interaction of the hollow particle with either a constituent particle of the artificial immune cell with a lytic enzyme as a part of its coating or natural immune cell possessing such lytic enzyme in which free radicals or reactive species of the oxidant are utilized to degrade the closure, seal or trap of the hollow particle. Specifically, the hollow core 60 of the particle contains sequestered biologically active molecules such as drugs or factors such as radioactive atoms or nanoparticles 61 that are prevented from escaping from the hollow particle by a closure, seal or trap 62 attached to the external aperture of the internal cavity of the particle. Free radicals or reactive species 18 of the oxidant 17 are generated in Fenton-type reactions catalyzed by the catalytic site 14 of the hollow core 60 and the same free radicals or reactive species 18 are utilized in the enzymatic reaction producing waste products 80 from the free radicals or reactive species 18; with the said reaction being catalyzed by the enzyme 66 attached to the artificial or natural immune cell 65.

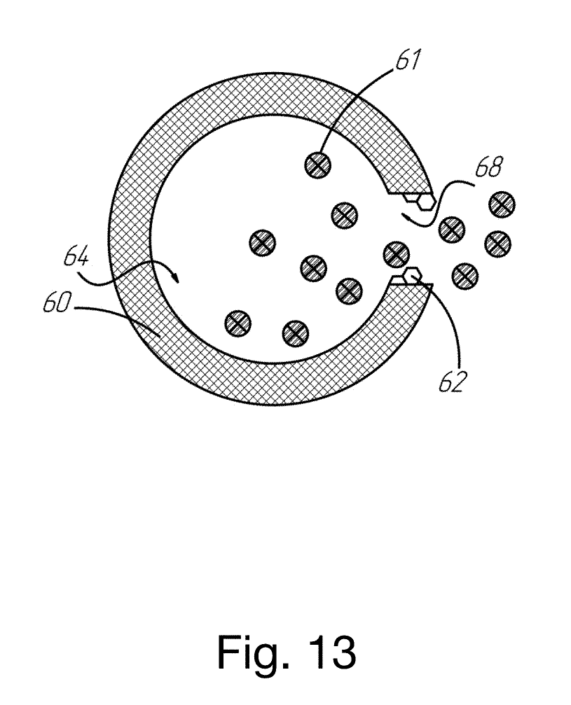

FIG. 13 depicts the degradation of the closure, seal or trap 62 as a result of the action of free radicals or reactive species as catalyzed by the enzyme attached to the artificial or natural immune cell as illustrated by FIG. 12 with the consequent opening of the aperture 68 and release of the previously sequestered molecules or factors 61 from the hollow core particle 60.

A characteristic embodiment of the depicted emulation of the property of the natural immune cells known as degranulation would be the incorporation of two varieties of particles into the artificial immune cell: the first species with hollow reactive core saturated with a desirable compound, e.g., an anti-tumor drug retained within the cavity of the core by a closure, seal or trap fashioned from carbon allotropes (graphene, carbon nanotubes) and the second particle containing a core localizable to the first particle via magnetic phenomena or specific immune binding and said second particle presenting with the enzyme myeloperoxidase attached to it. Upon localization of the artificial immune cell in the target tissue, the two constituent particles come in contact and, as free radicals 18 are generated from oxidants 17 on the reactive cores of the first species, these free radicals enter the enzymatic reaction catalyzed by myeloperoxidase that produces reactive species of chlorine capable of degradation of the graphene or carbon nanotubes, thus destroying the closure and opening the aperture 68 and allowing diffusion of the compound of interest 61 from the hollow cores of the first particle. Other materials that are easily degraded by oxidation such as proteins, glycoproteins, lipoproteins, nucleic acids, polymers of carbohydrates and enzymes can be utilized to facilitate free-radical driven and enzymatically catalyzed decomposition of the closure, seal or trap 62 and release of the trapped compounds 61.

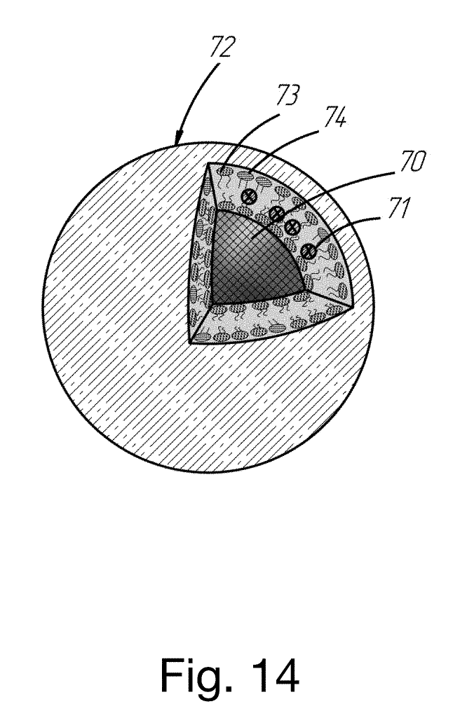

FIGS. 14, 15, 16, 17, 18, and 19 illustrate a different embodiment of the disclosed invention in which the artificial immune cell emulates degranulation of a natural immune cell. In this implementation the constituent particle of the artificial immune cell is a solid core 70 type of particle with coating 72 consisting of a bilayer of lipids 73 and 74 with biologically active molecules or factors 71 sequestered by intercalation between the two layers of the bilayer 72. FIG. 14 depicts the particle, its coating and the sequestered molecules such as drugs or factors such as radioactive atoms or nanoparticles as a pseudo-3D drawing.

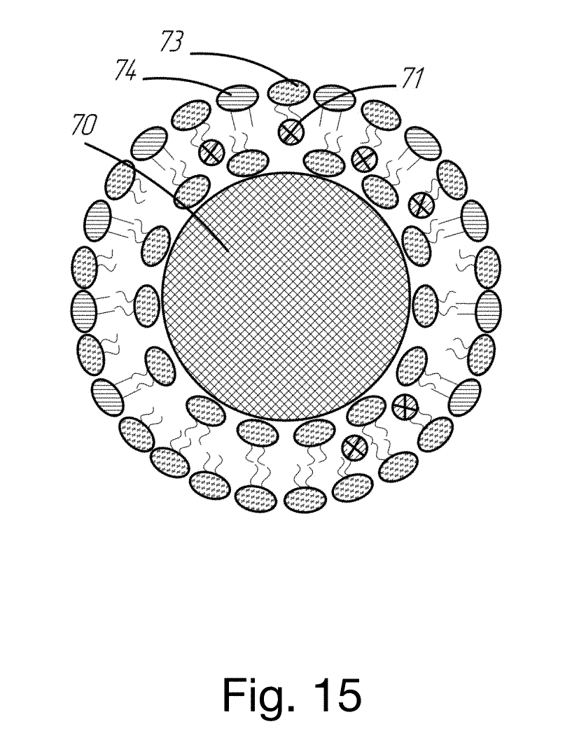

FIG. 15 presents the cross-section of the same particle revealing the solid magnetotactic core 70, the bilayer coating consisting of lipids 73 and 74 and biologically active molecules such as drugs or factors such as radioactive atoms or nanoparticles 71 sequestered in the coating via intercalation between the two layers of lipids 73 and 74.

Of note is the intended difference between the constituents of the bilayer: constituent lipid 73 is both capable of forming and sustaining the molecular bilayer and is not susceptible to lipid peroxidation chain reaction or other types of free radical induced dissociation of the bilayer while constituent lipid 74 while capable of forming and sustaining the molecular bilayer is specifically susceptible to lipid peroxidation chain reaction or other types of free radical induced dissociation of the bilayer.

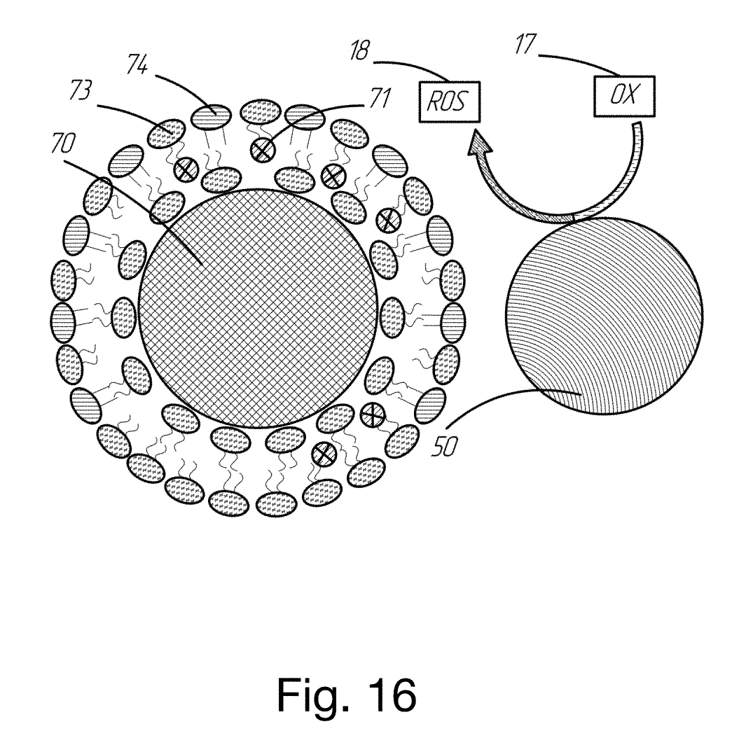

FIG. 16 illustrates the artificial immune cell comprised of two particles, the coated particle with solid magnetotactic core 70 and coating consisting of lipids 73 and 74 with intercalated sequestered molecules or factors 71 and the simple particle with catalytic and magnetotactic core 50 that is depicted as catalyzing the Fenton-type reaction in which initial oxidant 17 is catalytically transformed into free radicals or reactive species 18. As both cores are magnetotactic, they are either brought together by an external magnetic field or by residual magnetization after the external magnetic field has been terminated or shielded (not depicted).

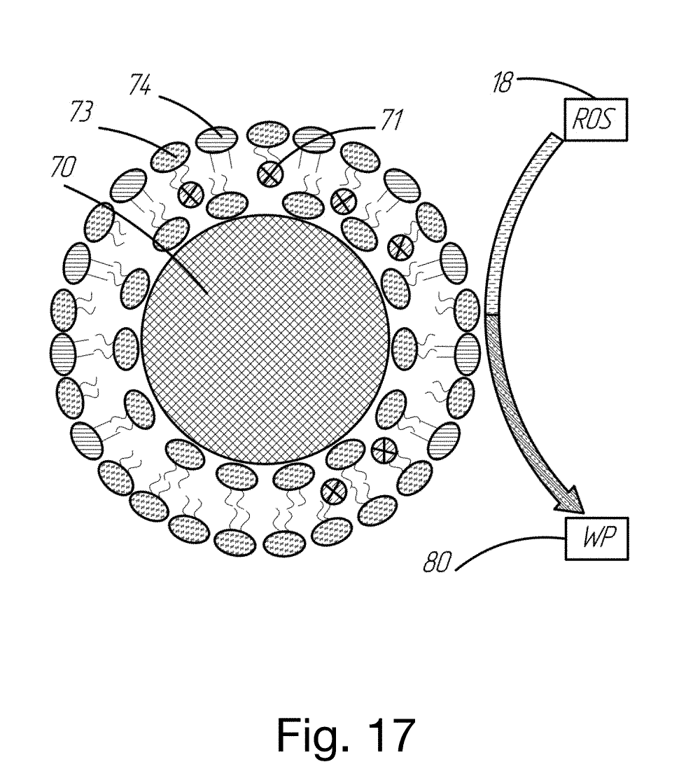

FIG. 17 illustrates the attack of free radicals or reactive species 18 on the lipid molecules 73 and 74 with the reactive species 18 transformed into waste products 80.

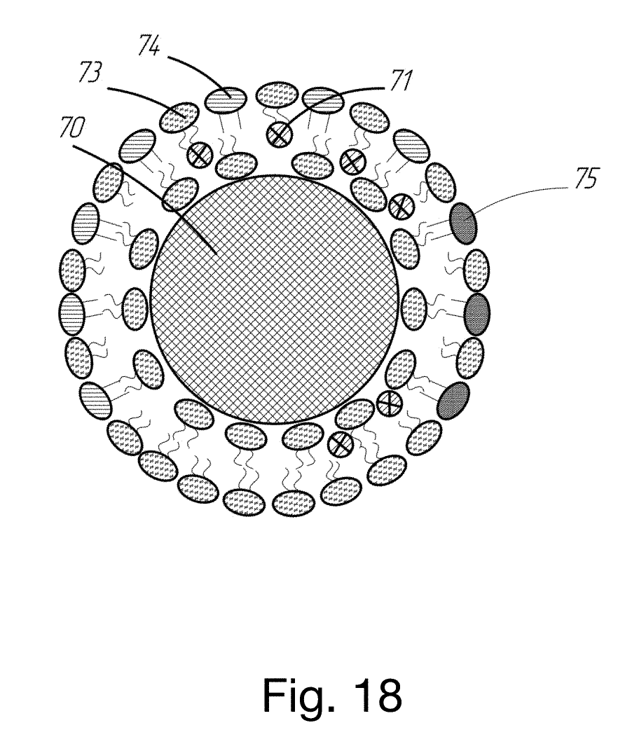

FIG. 18 illustrates the consequence of the oxidative insult caused by free radicals or reactive species 18 with lipids 74 undergoing degradation into lipids 75 via lipid peroxidation and becoming incapable of sustaining the molecular bilayer's integrity and mechanical properties.

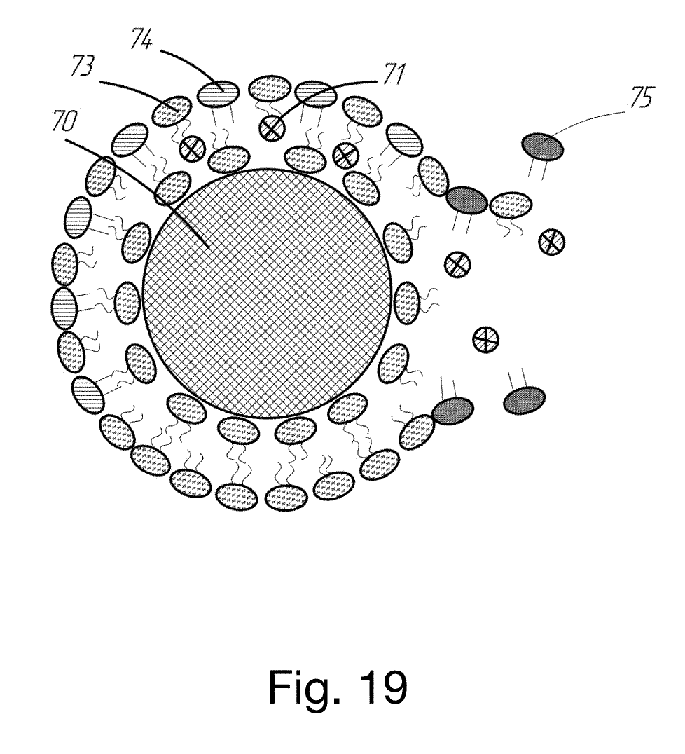

FIG. 19 depicts the partially disintegrated molecular bilayer with lipids 75 detaching from lipids 73 and 74 leading to liberation of the previously sequestered molecules or factors 71 and their diffusion away from the core 70.

In one of the embodiments of the disclosed invention lipid coating for the core 70 is derived from biologic particles such as lipoproteins of various density, including chylomicrons, VLDL, LDL, IDL and HDL particles, commonly known as blood lipid particles. Of note is that FIGS. 14-19 illustrate the schematics of this embodiment in a simplified fashion, since the actual geometry of the particle and its coating is mutable depending on the specifics of the manufacturing of said particles.

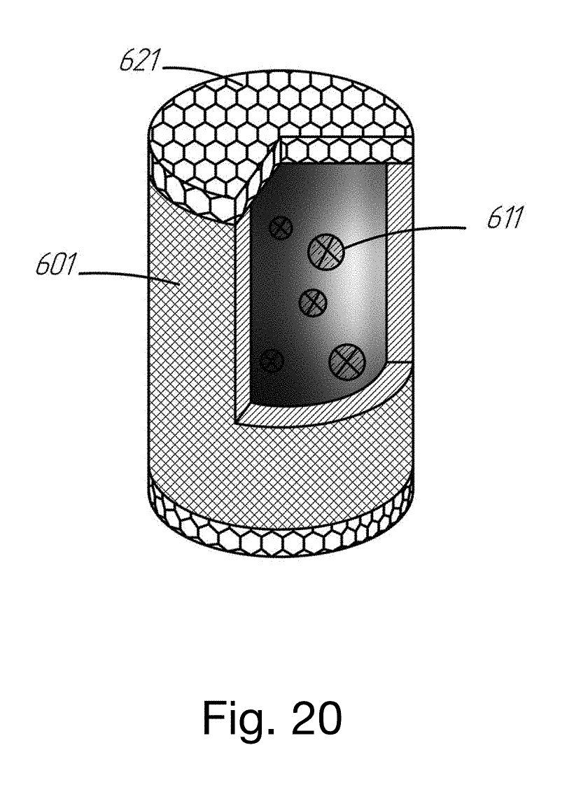

FIGS. 20, 21, and 22 illustrate a different embodiment of the disclosed invention in which the artificial immune cell is designed to emulate the property of a specific type of the natural immune cell, namely, the neutrophil. Said neutrophil is capable of releasing of extended web-like structures known as neutrophil extracellular traps consisting of strands of nucleic acid and globular proteins generated in the process of autolysis of chromatin and capable of extending in length to hundreds of nanometers while forming nets that bind, disarm, and kill microbes. In this embodiment of the artificial immune cell designed to emulate the aforementioned property of neutrophils, a specific type of the constituent particle is utilized as illustrated by FIGS. 20, 21, and 22.

FIG. 20 depicts the general view drawn in pseudo-3D of the constituent particle of the artificial immune cell capable of emulating the property of neutrophils such as production of the neutrophil extracellular traps. The core of said particle is a hollow cylinder 601 that is neither ferromagnetic nor ferrimagnetic but electroconductive and capped on both ends by structures 621 that function as closures or seals of the internal cavity of the hollow cylinder 601. Inside the hollow cylinder 601 are sequestered granules 611 of nucleic acid and protein mixture similar to that of the neutrophil extracellular trap.

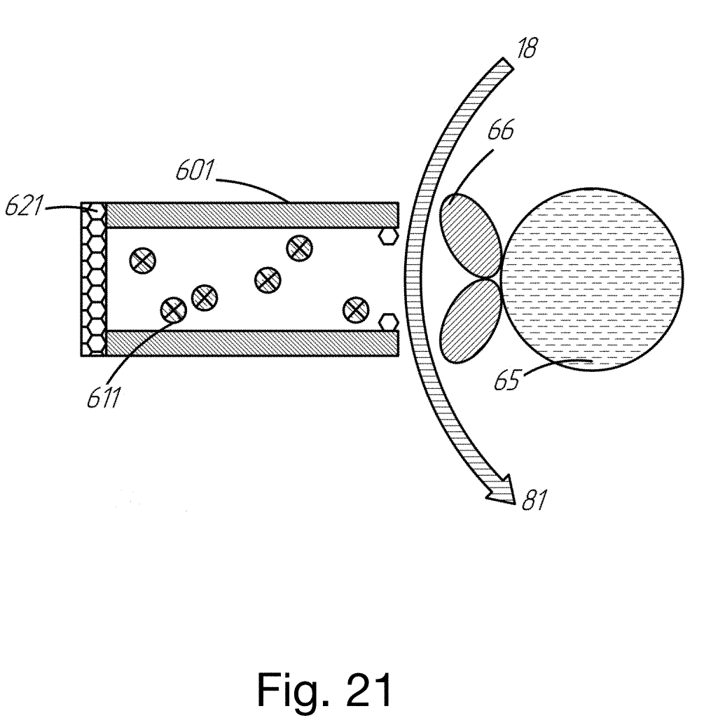

FIG. 21 illustrates the oxidative decomposition of the closure or seal structure 621 as a result of the oxidative attack of the free radicals or reactive species 18 catalyzed by the enzyme 66 such as myeloperoxidase or a similar enzyme present on 66, a natural immune cell or on a constituent particle of an artificial immune cell. Of note is the fact that only one side of the hollow cylinder particle is in the immediate vicinity of the enzyme 66, as the other side is hidden and shielded by other constituent particles of the artificial immune cell (particles not shown).

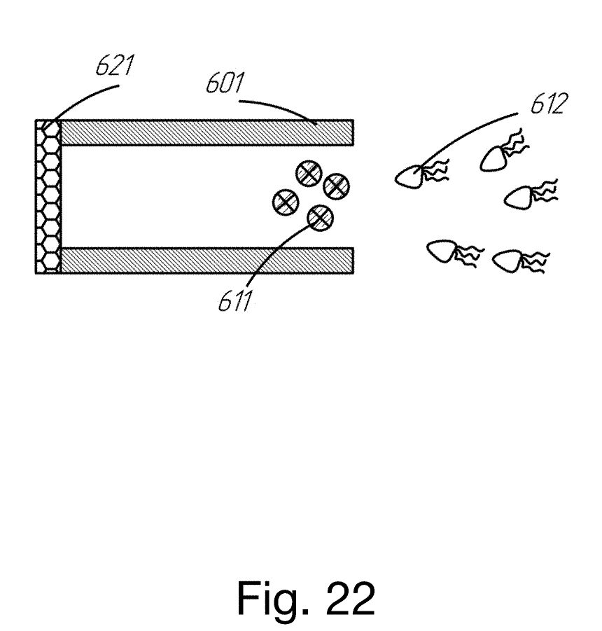

FIG. 22 depicts the ejection of the previously sequestered granules 611 and their unfolding into strands of nucleic acids and proteins 612 that form the emulated equivalent of the neutrophil extracellular trap. Such ejection may be due to the expected spring-like mechanical properties of the nucleic acids or it may be assisted by the application of external electromagnetic fields causing generation of eddy currents in the hollow cylinder 601 with subsequent electrostatic repelling of the charged globules and molecules of nucleic acids as well as heating of the hollow cylinder 601 and ejection of the globules and molecules of nucleic acids by the action of thermal expansion.

As noted above, the artificial immune cell may embodied as a construct or an aggregate of the constituent particles.

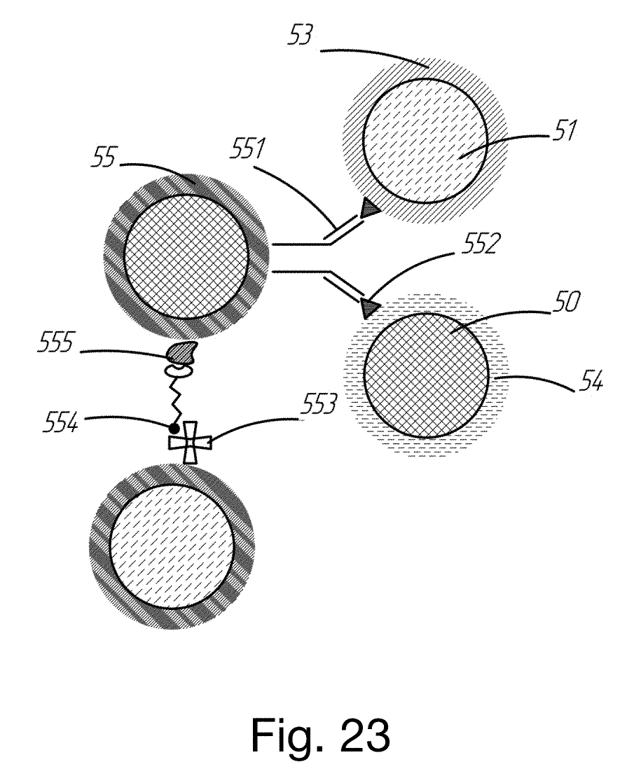

FIG. 23 illustrates the artificial immune cell whose constituent particles are held together with chemical bonds. Depicted are four constituent complex particles with cores 50 and 51 and coatings 53, 54, and 55. One of the particles with core 50 and coating 55 is decorated with functional molecules such as an immunoglobulin antibody 551 and a protein 555 with biotin moiety 554 attached to it. Another particle with core 50 and coating 54 is decorated with an antigen moiety 552 for which the antibody 551 is specific. Similarly, the third particle, with core 51 and coating 53 is decorated with the same antigen 552 for which the antibody 551 is specific. The forth particle with core 51 and coating 55 is decorated with streptavidin molecule 553. As depicted, the artificial immune cell is the construct of four constituent particles linked by chemical bonds, that of antigens 552 to antibody 551 and that of the biotin moiety 554 to streptavidin molecule 553. Only specific decorations participating in the forming of the construct are depicted and it is understood that other functional groups and molecules may be present on the surface of the constituent particles as well as aggregated with or within the construct. Of note is the use of streptavidin which may be substituted with avidin or other biotin-binding proteins and molecules. Of note is the fact that the depicted construct is characterized by the mechanical rigidity of the antibody-antigen bond and mechanical flexibility of the biotin-streptavidin bond as the reactive biotin moiety is attached to the protein 555 via a flexible chain of carbon atoms with single bonds.

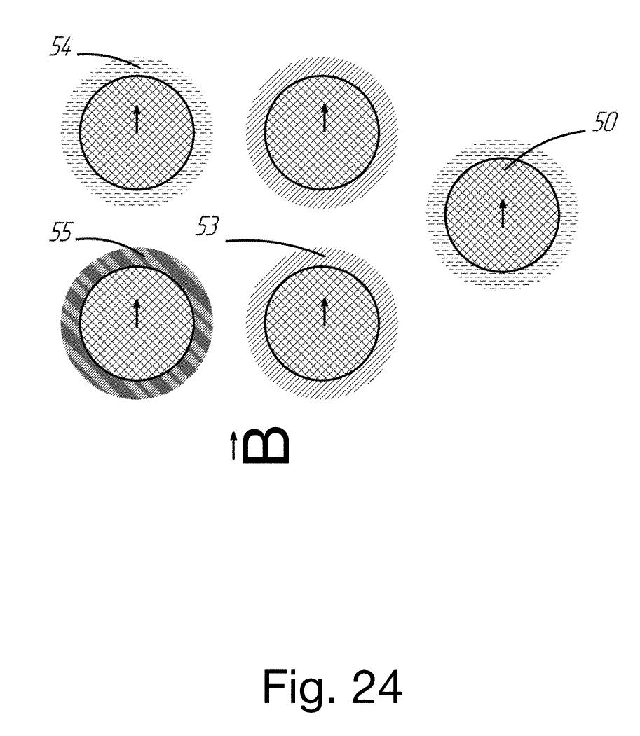

Alternately, the artificial immune cell may be an aggregate whose constituent particles are held together by means other than chemical bonds. FIG. 24 depicts such an aggregate of five constituent particles, all with cores 50 and varying coatings 53, 54, and 55 subjected to external magnetic field indicated by the vector B. As the cores 50 of the constituent particles are superparamagnetic, they align and aggregate under the influence of the external magnetic field.

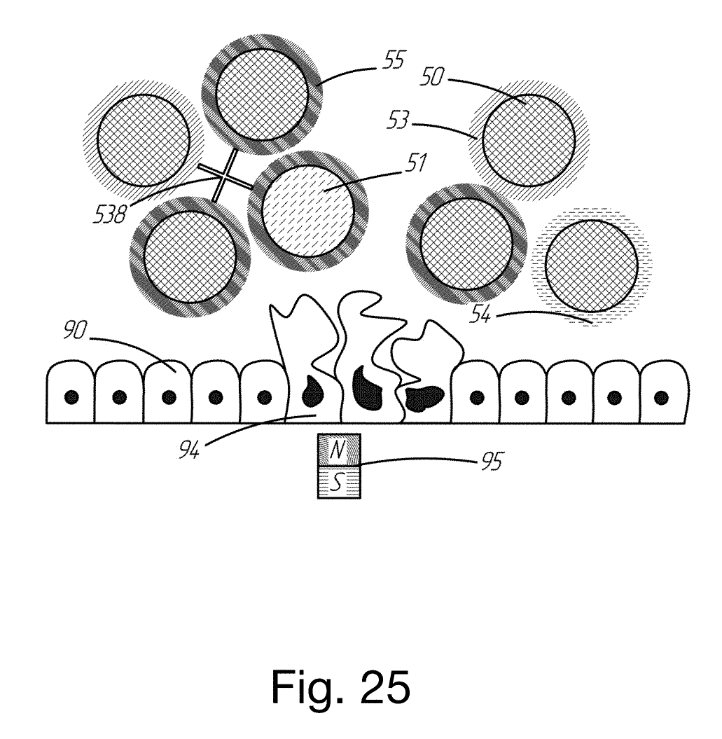

FIG. 25 depicts the schematic of one of the embodiment of the disclosed invention in which both types of artificial immune cell, that of a construct and that of an aggregate are present simultaneously in the biological target which in the depicted case is a conglomerate of non transformed (healthy) epithelial cells 90 with three cells 94 that are transformed (cancerous or malignant). The artificial immune cell that is a construct consists of four cores, of which three are of type 50 and one is of type 51, with all four cores coated with coatings types 55 and 53. The four constituent particles of the artificial immune cell that is a construct are held together by chemically bonded molecules 538, such as molecules of biotin and streptavidin or any other molecules capable of formation of strong and specific bonds. The artificial immune cell that is an aggregate depicted as consisting of three constituent particles with cores 50 and coatings 53, 54, and 55. As cores 50 possess magnetic reactivity they are magnetotactic.

Additionally, the emulation of the property of the natural immune cells known as adaptive immune competence requires modulation and termination of the activity of the artificial immune cell and its constituent particles. Such modulation and termination is achieved by introduction by application, injection, infusion, insufflation and similar means of soluble or particle-bound enzymatic compounds such as catalase, compounds that inhibit Fenton-type reactions, antibodies against cores and coatings of the constituent particles of the artificial immune cell, chelating agents and binding agents such as streptavidin into the biochemical milieu of the artificial immune cell. Mechanical, magnetic, electric and thermal deactivation and disassembly or disaggregation of the artificial immune cell is also useful and can be combined with the biochemical means of modulation and termination of activity of the artificial immune cell and may be followed by mechanical removal of the constituent particles such as by utilization of a magnetic trap or a magnetized filter.

The disclosed artificial immune cell(s) may be used for treatment of various human and animal diseases and conditions, such as cancer, autoimmune conditions and infections, including tuberculosis, as well as promotion of wound healing and tissue regeneration.

The disclosed invention is most relevant to the treatment of solid tumors, and specific bacterial infections (such as those caused by M. tuberculosis); however, with modifications, such as use of biologically active molecules in the coating, it may be used for disseminated cancers such as lymphomas and leukemias, as well as disseminated infections and sepsis.

It is known that cancerous cells may be induced to apoptosis and/or necrosis by high concentrations of free radicals, which is one of the benefits of the disclosed invention. Additionally, as the first target of free radicals released in the Fenton-type reactions by said particles will be the blood vessels, especially capillaries of the solid tumors, the damage to such blood vessels may lead to obturation of blood vessels with subsequent starvation of the tumor due to the reduction of blood flow through it, which may additionally reduce the formation of metastases.

It is also known that circulating immune complexes, often associated with autoimmune diseases, are susceptible to deactivation through oxidation by reactive oxygen species.

It is also known that certain specific Fenton-type reactions, such as iron-ascorbic acid mediated generation of hydroxyl radical are especially bactericidal in regard of Mycobacterium tuberculosis. With this in mind, the specific implementation aimed at treatment of M. tuberculosis infection may employ, among others, species of microscopic particles coated with lipid substances so formulated as to enhance the likelihood of adherence to the cell wall of the bacterium or antibodies to specific components of the mycobacterial cell wall, e.g., lipoarabinomannan.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes, which come within the meaning and range of equivalency of the claims are to be embraced within their scope.

Although preferred embodiments of the invention have been described herein in detail, it will be understood by those skilled in the art that variations may be made thereto without departing from the spirit of the invention or the scope of the appended claims.

### REFERENCE SIGNS LIST

10—compound of a transition or post-transition metal, such as Fe, Cu, Cr, Co, V, Ti, Ni, Ga, In, et alii;
11—compound of a transition or post-transition metal in its reduced form, such as Fe3O4;
12—compound of a transition or post-transition metal in its oxidized form, such as Fe2O3;
14—catalytic site containing atoms, ions, oxides or other compounds of a transition or post-transition metal;
17—compound serving as the oxidant;
18—compound that is a free radical or a reactive species form of the oxidant;
21—compound serving as the antioxidant;
22—compound that is the oxidized form of the antioxidant;
50—microscopic core containing one or more compounds of one or more transition or post-transition metals (catalytic core) with or without magnetotactic properties;
51—microscopic core that does not contain compounds of transition or post-transition metals (non-catalytic core);
52—outer coating containing one or more compounds capable of inhibition of Fenton-type reactions;
53—outer coating containing antioxidant chemicals;
54—outer coating containing oxidizers or pro-oxidant chemicals;
55—outer coating containing biologically active molecules;
551—immunoglobulin antibody;
552—antigen moiety for which the antibody 551 is specific;
553—streptavidin or avidin molecule;
554—biotin molecule;
555—protein attached to coating 55;
538—bonded molecules connecting the component particles of the artificial immune cell construct;
60—hollow core particle containing one or more compounds of one or more transition or post-transition metals;
61—compounds or particles of interest sequestered within the cavity of the hollow core;
62—layered compound sealing the cavity of the hollow core, forming a seal, closure or a trap;
64—internal cavity of the hollow core particle;
65—a natural immune cell or a constituent particle of an artificial immune cell;
66—molecule of an enzyme;
68—aperture in the layered compound trap 62 opened by enzyme 66;
601—hollow cylinder core that is not magnetic but electroconductive;
611—mixture of nucleic acids and proteins;
621—closure, seal or trap susceptible to degradation under oxidative insult;
70—core of the particle possessing magnetotactic and catalytic properties;
71—compounds or factors sequestered within the molecular bilayer 72;
72—molecular bilayer;
73—molecules capable of forming and sustaining the molecular bilayer and not susceptible to lipid peroxidation chain reaction or other types of free radical induced dissociation of the bilayer;
74—molecules capable of forming or sustaining the molecular bilayer and susceptible to lipid peroxidation reaction or other free radical induces dissociation of the bilayer;
75—molecules no longer capable of sustaining the molecular bilayer due to lipid peroxidation or other free radical induced chemical change;
80—waste products derived from reactive oxidizing species 18;
90—non-transformed (healthy) epithelial cells;
94—transformed (cancerous) cells;
95—permanent or electric magnet.

### CITATIONS LIST

### Patent Literature

U.S. Pat. No. 8,088,358 Filing date: Mar. 4, 2002 Publication date: Jan. 3, 2012 Applicant: Centrum Fur Angewandte Nanotechnologie (Can) Gmbh Title: Paramagnetic nanoparticle
U.S. Pat. No. 9,259,468 Filing Date: Oct. 9, 2012 Publication date: Feb. 16, 2016 Applicant: University of Central Florida Research Foundation, Inc. Title: Cancer therapy via selective uptake of specialized nanoparticles in cancer cells
U.S. Pat. No. 8,651,113 B2 Filing date: Jul. 17, 2009 Publication date: Feb. 18, 2014 Applicant: Swr&D Inc. Title: Magnetically responsive nanoparticle therapeutic constructs and methods of making and using

### Non-Patent Literature

Koppenol W H. (2002) The Haber-Weiss Cycle—71 Years Later. Redox Report, 7(1), 59-60.
Nies D H, Silver S, eds. Molecular Microbiology of Heavy Metals. Springer (2007), pp. 80-82.
Wardman P, Candeias L P. (1996) Fenton Chemistry: An Introduction. Radiation Research; 145(5), 523-531.
Burkitt M K, Gilbert B C. (1990) Model Studies of the Iron Catalyzed Haber-Weiss cycle and the ascorbate-driven Fenton Reaction. Free Radical Research Communications, 10(4-5), 265-280.
Lemire J A et al. (2013) Antimicrobial activity of metals: mechanisms, molecular targets and applications. Nature Reviews Microbiology, 11: 371-384.

What is claimed and desired to be secured by United States Letters Patent is:

1. An artificial immune cell as a construct or aggregate consisting of several microscale or nanoscale particles either of identical composition and properties or several differing compositions and properties that upon introduction into or onto a biologic target presents with properties similar to those of intrinsic immune cells known as granulocytes inclusive of but not limited to:
the necessarily and essential to the disclosed invention ability to generate free radicals and reactive species such as those of oxygen, nitrogen, halogens such as fluorine, chlorine, bromine, iodine and astatine, other inorganic and organic free radicals and reactive species by the means of Fenton and Fenton-type reactions catalyzed by the construct itself or its constituent components, recognized as an established property of the intrinsic immune cells whereas said free radicals or reactive species serve as communication and mediation molecules or, when released in large quantities, as toxic factors;
the necessarily and essential to the disclosed invention ability for spontaneous as well as controlled locomotion or motility defined as the spontaneous but essential ability to follow an outside flow or movement in a passive fashion or to move in a controlled and directed fashion being controlled and directed either by outside forces such as electromagnetic fields, energetic waves or any combination of the two or inside forces such as changes of composition and gradients present in the biochemical milieu that said construct is immersed into but preferably both either simultaneously or one at a time; recognized as being similar to motility of the intrinsic immune cells including granulocytes;
the optional but desirable in certain implementations of the disclosed invention ability to identify and strongly and specifically bind to specific targets inclusive of antigens, other molecules, intracellular and extracellular structures, specific cells, microscopic organisms, tissues, organs, tumors, wounds, biologic systems and multicellular organisms; recognized as being similar to the property of the intrinsic immune cells known as specificity of immune recognition observed in multiple varieties of immune cells including granulocytes;
the optional but desirable in certain implementations of the disclosed invention ability for controlled release and targeted delivery of previously sequestered molecules, including biologically active molecules and factors as well as molecules and factors that are capable of initiating, sustaining or terminating of various chemical reactions, that may be contained within the constituent particles of the artificial immune cell construct or aggregate or between the constituent particles of the artificial immune cell construct or aggregate, with said ability recognized as being similar to the degranulation and neutrophil extracellular trap production processes specific to the intrinsic immune cells known as granulocytes;
the necessary and essential to the disclosed invention ability to amplify, reduce, modulate and terminate the biologic and chemical activity including but not limited to generation of free radicals, controlled and spontaneous locomotion, specific identification and binding to targets as well as release of previously sequestered molecules with said ability recognized as being similar to the property of responsiveness and modulation of response as well as reversible termination of response and irreversible termination of activity due to cellular death with all of these being properties of the intrinsic immune cells including granulocytes.

2. The artificial immune cell as defined in claim 1 consisting of a construct defined as several constituent microscale or nanoscale particles linked together by chemical bonds arising from separate microscale or nanoscale particles in the process of self-assembly mediated by chemical reactions or consisting of an aggregate of several constituent microscale or nanoscale particles brought together by outside forces such as electromagnetic fields but not linked together by chemical bonds.

3. The artificial immune cell as defined in claim 1 whereas the microscale constituent particles are defined as particles with sizes ranging from several micrometers to several hundred micrometers and the nanoscale constituent particles defined as particles with sizes ranging from fractions of a nanometer to several hundred nanometers with the consideration that microscale particles are utilized in the cases when obturation of blood and lymph vessels and extracellular spaces is not to be avoided and the processes of extravasation and endocytosis are not essential or outright undesirable and the consideration that nanoscale particles are utilized in the cases when obturation of blood and lymph vessels and extracellular spaces is to be avoided and the processes of extravasation and endocytosis are essential or desirable.

4. The artificial immune cell as defined in claim 1 whereas the composition of the constituent particles is defined as the chemical identity of said particles and their components, with the chemical identity selected from pro-oxidant compounds such as hydrogen peroxide, chemically bound ground state triplet oxygen and singlet oxygen such as to hemoglobin, myoglobin, hemocyanin and similar molecules, solutions of oxygen and singlet oxygen in solids and liquids including but not limited to fluorocarbon compounds such as octadecafluorodecalin (perfluorodecalin) and the like; homogenous and heterogenous catalytic compounds capable of sustaining Fenton and Fenton-type reactions such as oxides and other compounds of copper, iron, nickel and other transition and post-transition metals as well as heterogeneous and insoluble catalysts including but not limited to layered alumosilicates and carbon nanotubes; anti-oxidant compounds including human vitamins C (ascorbic acid) and E (mixed tocopherols and tocotrienols), human intrinsic antioxidants such as ubiquinol (coenzyme Q), alpha-lipoic acid, glutathione, lutein and other carotenoids, polyphenolic compounds such as resveratrol and other antioxidants.

5. The artificial immune cell as defined in claim 1 whereas the properties of the constituent particles include geometric shape and geometric arrangement of the particles and their components with the shapes of the particles and their components being spherical, cylindrical, cubic, that of a parallelepiped and irregular, and the geometric arrangement of the components of the particles being that of a core surrounded by coating, hollow core with an aperture closed by a cap, hollow cylinder capped on one or both ends and other suitable arrangements.

6. The artificial immune cell as defined in claim 1 whereas the introduction into or onto a biologic target is accomplished by topical application, injection, infusion, electrophoresis, iontophoresis, magnetophoresis and similar means together with such passive means of delivery including diffusion, delivery with blood flow, lymph flow, interstitial fluid flow, peristaltic flow, cerebrospinal fluid flow, flow of tissue and organ secretions, intracellular flow and microtubule-based delivery, and movement along a gradient such as a gradient of pH or any other suitable gradient.

7. The artificial immune cell as defined in claim 1 whereas the ability to generate free radicals and reactive species is facilitated by said particles and their constituent components such as cores, coatings, and attached chemical and biologically active compounds that participate in specific chemical reactions known as Fenton reaction and Fenton-type reactions including the Haber-Weiss reaction by supplying consumed chemical compounds, catalytic compounds and reaction-modulating compounds, allowing the said reactions to proceed in a self-sustaining cyclic fashion in which the first part of the cycle sees the reduced form of the catalytic compound such as a transition metal or post-transition metal compound inclusive of ions, oxides and other compounds of elements of the Groups 3 (IIIB) through 12 (IIB) of the Periodic table including but not limited to iron, copper, nickel, cobalt, ruthenium, gold and others react with the oxidizing compound producing the oxidized form of the catalytic compound and a different oxidant, derived from initial oxidant and generally viewed as the more aggressive free radical or reactive species and the second part of the cycle sees the oxidized form of the catalytic compound react with an antioxidant compound thus regenerating the catalytic compound to its reduced form and producing a stable radical or oxidized form of the antioxidant generally viewed as a waste product; whereas the consumed chemical compounds include oxidants such as hydrogen peroxide, lipid peroxides, elemental or molecular oxygen, radicals of oxygen such as superoxide radical, radicals of nitrogen such as nitric oxide or peroxynitrite, compounds of halogens such as hypochlorous acid and the like, exogenous pro-oxidants such as menadione (vitamin K3, C₁₁H₈O₂), antibiotics such as doxorubicin (C₂₇H₂₉NO₁₁) and bleomycin (C₅₅H₈₄N₁₇O₂₁S₃), toxins inclusive of paraquat (C₁₂H₁₄Cl₂N₂), and potassium triiodide (I₂KI) as well as other suitable compounds capable of entering into Fenton-type reactions, including complex systems relying on endogenous enzymes such as NADPH oxidase, xanthine oxidase, myeloperoxidase and monoamine oxidase that generate free radicals in the presence of molecular oxygen or other oxidant substance; whereas the consumed chemical compounds include antioxidants such as ascorbic acid (vitamin C), species of tocopherols and tocotrienols (vitamin E), reduced forms of cofactors such as nicotinamide adenine dinucleotide (NADH), nicotinamide adenine dinucleotide phosphate (NADPH), flavin adenine dinucleotide (semiquinone FADH and hydroquinone FADH2), uric acid, ubiquinol (coenzyme Q), alpha lipoic acid, polyphenols, flavans and flavonoids, as well as substantially similar compounds; whereas the produced free radicals and reactive species include hydroxyl radical .OH, superoxide radical .O₂—, hydrogen peroxide, nitric oxide, peroxynitrite, oxybenzone, tyrosyl radical, peroxyl and alkoxyl radicals as well as compounds of halogens and the like.

8. The artificial immune cell as defined in claim 1 whereas the ability of said particles for controlled locomotion is facilitated by the constituent components of said particles such as cores, coatings, and attached compounds that exhibit magnetotactic properties as observed in magnetic materials exhibiting paramagnetism, ferromagnetism, ferrimagnetism and especially superparamagnetism as observed in small particles of ferromagnetic or ferrimagnetic materials with geometric sizes and shapes similar or smaller to the size of the single magnetic domain, with such materials as iron oxides (magnetite, Fe3O4 and maghemite, gamma-Fe2O3), mixed ferrites containing iron, oxygen and other elements such as copper, cobalt, nickel, ruthenium, gold and other transition and post-transition metals and their chemical compounds being preferred that are propelled by external magentic fields and, optionally, implanted or applied permanent magnets and electromagnets.

9. The artificial immune cell as defined in claim 1 whereas the ability of said particles for controlled locomotion is optionally facilitated by the attachment of said particles to intrinsically motile biologic objects such as motile bacteria, immune cells, and spermatozoa whereas such motile biologic objects propel themselves and the attached particles utilizing the activity of flagella, pseudopodia, cytoskeletal filaments, cilia and pili to produce directional movement guided by the presence and gradients of electromagnetic fields, gradients of chemical nature such as gradients of pH, ionic and non-ionic compounds serving as biochemical attractors and other means of directing and controlling the intrinsic motility of such biologic objects.

10. The artificial immune cell as defined in claim 1 whereas the ability of said particles to identify and strongly and specifically bind to specific targets is facilitated by the inclusion into said particles of recognition and binding components and compounds such as antibodies capable of recognizing and binding to specific antigens, fragments of complement proteins such as C1q, C4b and C3b that recognize and bind the Fc portion of an antibody molecule when bound to an antigen, as well as strong binding observed in the biotin-streptavidin and biotin-avidin systems whereas the either the biotin moiety or the (strept)avidin molecule are attached to the particle or a component of the particle, preferably to the outer and easily accessible components of the particle such as its coating.

11. The artificial immune cell as defined in claim 1 whereas the ability for controlled release and targeted delivery of previously sequestered inside the constituent particles biologically active molecules including nucleic acids, proteins, drugs and factors including radioactive atoms or particles is facilitated by degradation of specific components of the particles such as coatings, seals, traps or closures by the action of free radicals generated in Fenton and Fenton-type reactions with or without additional chemical reactions mediated by the present biological objects such as intrinsically present natural immune cells or their enzymes and other constituents with the biologically active molecules or factors passively diffusing through the degraded coatings, seals, traps or closures or actively expelled by the electrostatic, thermal or magnetic interactions with preferred embodiments utilizing molecular bilayers in the form of a coating deposited on the surface of the particle from phospholipids or similar compounds and degraded through the free radical mediated lipid peroxidation chain reactions that disrupt the bilayer allowing for biologically active molecules or factors previously sequestered between the layers of the bilayer to escape or, alternately, degradation of seals, traps and closures attached to the apertures of the internal cavities of spherical or irregularly shaped particles or as capping seals attached to one or both ends of cylindrical and similarly shaped particles and consisting of chemical compounds that are easily degraded by oxidation such as proteins, glycoproteins, lipoproteins, nucleic acids, polymers of carbohydrates and especially carbon allotropes such as carbon nanotubes and graphene sheets that are degraded by the action of the enzyme myeloperoxidase in the presence of reactive species of oxygen, as well as other similar compounds whose degradation leads to opening of apertures and diffusion or ejection of the previously sequestered biologically active molecules and factors.

12. The artificial immune cell as defined in claim 1 whereas the ability to amplify, reduce, modulate and terminate the activity of said artificial immune cell is attained by variation of the physical factors and biochemical milieu of said artificial immune cell inclusive but not limited to mechanical removal of the constituent particles by electromagnetic fields via magnetotactic effects, electrical currents and electrostatic potentials applied to the constituent particles; inclusive but not limited to deactivation of Fenton and Fenton-type reactions by removal or chemical binding of the catalytic components of constituent particles such as by binding of iron atoms by deferoxamine, deferasirox, deferiprone and similar chelating agents, binding of copper atoms by penicillamine, trientine, tetrathiomolybdate, metallothionine or similar chelating agents, binding of cobalt and nickel atoms by N-acetyl-L-cysteine and other chelating agents;
inclusive but not limited to deactivation of Fenton and Fenton-type reactions by excess amounts of antioxidants capable of terminating the reactions such as nitric oxide, ascorbic acid, vitamin E, retinoids, carotenoids, and other such substances or delivery of particles coated with antioxidant enzymes such as superoxide dismutases, catalase, peroxiredoxins, as well as glutathione reductase, glutathione peroxidase and glutathione S-transferase supplied with additional reduced glutathione; inclusive but not limited to disruption of the artificial immune cell constructs or aggregates with electromagnetic fields, energetic waves such as ultrasound or by introduction of additional particles that bind and disrupt the artificial immune cell constructs or aggregates by crowding the active particles and reducing the availability of chemicals consumed in Fenton and Fenton-type reactions to said active particles; inclusive but not limited to introduction of localized hypoxia and other physical and chemical conditions that prevent further generation of free radicals and inclusive but not limited to induction or stimulation of phagocytosis of the constituent particles of the artificial immune cell by the intrinsically present immune cells such as phagocytes or, similarly, endocytosis of the constituent particles of the artificial immune cell by the biologic targets themselves such as cells, microorganisms, tissues, organs and the like.
